(19) United States
(12) Patent Application Publication
(10) Pub. No.: US 2006/0063736 A1
(43) Pub. Date: Mar. 23, 2006

(54) Compositions and methods for inhibiting mucin-type O-linked glycosylation

(76) Inventors: Carolyn R. Bertozzi, Berkeley, CA (US); Howard C. Hang, Somerville, MA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/088,546

(22) Filed: Mar. 23, 2005

Related U.S. Application Data
(60) Provisional application No. 60/556,673, filed on Mar. 25, 2004.

(51) Int. Cl. A61K 31/7072; C12Q 1/48; C07H 19/12; C12N 9/99

(52) U.S. Cl. 514/49; 536/28.3; 435/184; 435/15

(57) ABSTRACT

The present invention provides inhibitors of mucin-type O-linked glycosylation, and in particular inhibitors of polypeptide N-acetyl-α-galactosaminyltransferases; as well as compositions comprising the inhibitors. The present invention further provides methods of identifying inhibitors of polypeptide N-acetyl-α-galactosaminyltransferases. The inhibitors are useful in various applications, including research applications, and treatment methods.

### CROSS-REFERENCE

This application claims the benefit of U.S. Provisional Patent Application No. 60/556,673, filed Mar. 25, 2004, which application is incorporated herein by reference in its entirety.

### STATEMENT REGARDING FEDERALLY SPONSORED RESEARCH

The U.S. government may have certain rights in this invention, pursuant to grant no. GM66047 awarded by the National Institutes of Health.

### FIELD OF THE INVENTION

The present invention is in the field of modulators of mucin-type O-linked glycosylation, and in particular to modulators of polypeptide N-acetyl-α-galactosaminyltransferases.

### BACKGROUND OF THE INVENTION

Protein glycosylation is important for a variety of cellular events such as protein trafficking and cell-cell interactions. There are two major forms of protein glycosylation, N-linked and O-linked, distinguished by their glycosidic linkages to amino acid side chains. Mucin-type O-linked glycosylation is the dominant form of O-linked glycosylation in higher eukaryotes, characterized by an N-acetyl-α-galactosamine (GalNAc) residue attached to the hydroxyl group of serine or threonine side chains (FIG. 1). The biosynthesis of O-linked glycans is initiated by the family of polypeptide N-acetyl-α-galactosaminyltransferases (ppGalNAcTs), which transfer GalNAc from uridine diphosphate N-acetyl-α-galactosamine (UDP-GalNAc) onto proteins trafficking through the Golgi compartment (FIG. 1). Elaboration of the core glycopeptide, termed the Tn-antigen, by downstream glycosyltransferases affords more complex glycan structures. These O-linked glycans are thought to play critical roles in lubrication and protection of tissues, leukocyte homing, the immune response, and the metastasis of tumor cells.

While much is known about the functions of N-linked glycans, progress toward understanding O-linked glycosylation has been hindered by the large number of ppGalNAcT isoforms present in vertebrate genomes (˜24 in human). To date, 21 putative ppGalNAcTs have been cloned from various organisms, all of which have been biochemically characterized with the exception of ppGalNAcT-8. Transcript analysis has revealed differential tissue distribution and temporal regulation of ppGalNAcT expression during development and pregnancy. Mice deficient in ppGalNAcT-1, -4, -5, or -13 demonstrate no apparent phenotypes with respect to development, fertility and immune function, suggesting functional redundancy or compensatory regulation amongst the ppGalNAcT family members. However, recent studies of D. melanogaster mutants have demonstrated that one ppGalNAcT, pgant35A, is essential for development.

Studies of O-linked glycoprotein biosynthesis are further complicated by the overlapping peptide substrate specificities exhibited by the ppGalNAcT family in vitro and in vivo. The identification of ppGalNAcTs that specifically recognize α-GalNAc-modified glycopeptides has enabled further subclassification of the family into peptide and glycopeptide transferases. In contrast to N-linked glycosylation, where a single oligosaccharyl transferase catalyzes the modification of asparagine residues within the consensus sequence Asn-Xaa-Ser/Thr, no consensus sequence for O-linked glycosylation has been identified. Computational algorithms developed to predict the likelihood of O-linked glycosylation from primary amino acid sequences have been useful for identifying mucin domains within a protein. However, these semi-empirical methods have limited accuracy for predicting glycosylation of specific residues and therefore still require experimental confirmation. Finally, structural studies of acceptor peptide substrates suggest that ppGalNAcTs may recognize β-turn-like motifs rather than primary amino acid sequence alone.

The discovery and design of inhibitors that target N-linked glycan biosynthesis and processing have greatly increased our appreciation of N-linked glycosylation. In contrast, few chemical tools are available to address mucin-type O-linked glycosylation. Competitive substrate-based primers can be used to inhibit the downstream elaboration of O-linked glycans in cells, affording truncated structures. However, these compounds do not affect the attachment of GalNAc to Ser or Thr.

There is a need in the art for inhibitors of mucin-type O-linked glycosylation. The present invention addresses this need.

### LITERATURE

Van den Steen et al. (1998) Crit. Rev. Biochem. Mol. Biol. 33:151-208; Ten Hagen et al. (2003) Glycobiology 13:1-16; Winans and Bertozzi (2002) Chem. Biol. 9:113-129; Taylor-Papdimitriou et al. (1999) Biochem Biophys. Acta 1455:301-313; Tsuboi and Fukuda (2001) Bioessays 23:46-53; Lasky (1995) Annu. Rev. Biochem. 64:113-139; Kuan et al. (1989) J. Biol. Chem. 264:19271-19277; Sarkar et al. (1995) Proc. Natl. Acad. Sci. USA 92:3323-3327; Sarkar et al. (1997) J. Biol. Chem. 272:25608-25616; Brown et al. (2003) J. Biol. Chem. 278:23352-23359; Fuster et al. (2003) Cancer Res. 63:2775-2781; Ten Hagen et al. (1998) J. Biol. Chem. 273:27749-27754; Wragg et al. (1995) J. Biol. Chem. 270:16974-16954; Scherman et al. (2003) Antimicrob. Agents Chemother. 47:378-38.

### SUMMARY OF THE INVENTION

The present invention provides inhibitors of mucin-type O-linked glycosylation, and in particular inhibitors of polypeptide N-acetyl-α-galactosaminyltransferases; as well as compositions comprising the inhibitors. The present invention further provides methods of identifying inhibitors of polypeptide N-acetyl-α-galactosaminyltransferases. The inhibitors are useful in various applications, including research applications, and treatment methods.

### BRIEF DESCRIPTION OF THE DRAWINGS

FIG. 1 depicts initiation of mucin-type O-linked glycosylation by ppGalNAcTs and elaboration into complex O-linked glycans by downstream glycosyltransferases.

FIGS. 2A and 2B depict the design and synthesis of a uridine-based library as nucleotide sugar mimics.

FIGS. 3A-C depict an enzyme-linked lectin assay (ELLA) for detecting ppGalNAcT activity. FIG. 3A depicts the basic design of the assay. FIG. 3B depicts exemplary ppGalNAcT substrates peptide 4 (SEQ ID NO: 16) and glycopeptide 5 (SEQ ID NO:17). FIG. 3C depicts the results of an assay.

FIGS. 4A and 4B depict ppGalNAcT inhibitors identified from the uridine-based library. FIG. 4A depicts the structures of mppGalNAcT-1 inhibitors 1-68A and 2-68A and parent aldehyde 68A. FIG. 4B depicts K_{I} measurements for 1-68A, 2-68A and 68A with respect to UDP-GalNAc, against mppGalNAcT-1.

FIGS. 5A-D depict cellular effects of ppGalNAcT inhibitors and apoptosis inducers (doxorubicin and campothecin) in Jurkat cells.

### DEFINITIONS

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, at least 75% free, at least 85% free, at least 90% free, at least 95% free, or at least 98% free, or more, from other components with which it is naturally associated. A “substantially purified” compound is a compound that is at least 80% pure, at least 85%, at least 90% pure, at least 95% pure, at least 98% pure, or at least 99% pure, e.g., is free of components with which the compound may be naturally associated, or other undesirable components such as contaminants.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as certain lymphocyte populations, glial cells, macrophages, tumor cells, peripheral blood mononuclear cells (PBMC), and the like. The term “biological sample” encompasses a clinical sample, and also includes cells in culture, cell supernatants, tissue samples, organs, bone marrow, and the like.

The term “lower alkyl”, alone or in combination, generally refers to an acyclic alkyl radical containing from 1 to about 10, preferably from 1 to about 8 carbon atoms and more preferably 1 to about 6 carbon atoms. Examples of such radicals include methyl, ethyl, n-propyl, isopropyl, n-butyl, isobutyl, sec-butyl, tert-butyl, pentyl, iso-amyl, hexyl, octyl and the like.

“Alkyl” is a monovalent, saturated or unsaturated, straight, branched or cyclic, aliphatic (i.e., not aromatic) hydrocarbon group. In various embodiments, the alkyl group has 1-20 carbon atoms, i.e., is a C1-C20 (or C₁-C₂₀) group, or is a C1-C18 group, a C1-C12 group, a C1-C6 group, or a C1-C4 group. Independently, in various embodiments, the alkyl group: has zero branches (i.e., is a straight chain), one branch, two branches, or more than two branches; is saturated; is unsaturated (where an unsaturated alkyl group may have one double bond, two double bonds, more than two double bonds, and/or one triple bond, two triple bonds, or more than three triple bonds); is, or includes, a cyclic structure; is acyclic. Exemplary alkyl groups include C₁alkyl (i.e., —CH₃ (methyl)), C₂alkyl (i.e., —CH₂CH₃ (ethyl), —CH═CH₂ (ethenyl) and —C≡CH (ethynyl)) and C₃alkyl (i.e., —CH₂CH₂CH₃ (n-propyl), —CH(CH₃)₂ (i-propyl), —CH═CH—CH₃ (1-propenyl), —C≡C—CH₃ (1-propynyl), —CH₂—CH═CH₂ (2-propenyl), —CH₂—C≡CH (2-propynyl), —C(CH₃)═CH₂ (1-methylethenyl), and —CH(CH₂)₂ (cyclopropyl)).

“Ar” indicates a carbocyclic aryl group selected from phenyl, substituted phenyl, naphthyl, and substituted naphthyl. Suitable substituents on a phenyl or naphthyl ring include C₁-C₆alkyl, C₁-C₆alkoxy, carboxyl, carbonyl(C₁-C₆)alkoxy, halogen, hydroxyl, nitro, —SO₃H, and amino.

The term “aryl” as used herein refers to 5- and 6-membered single-aromatic radicals which may include from zero to four heteroatoms. Representative aryls include phenyl, thienyl, furanyl, pyridinyl, (is)oxazoyl and the like.

“Aryl” is a monovalent, aromatic, hydrocarbon, ring system. The ring system may be monocyclic or fused polycyclic (e.g., bicyclic, tricyclic, etc.). In various embodiments, the monocyclic aryl ring is C5-C10, or C5-C7, or C5-C6, where these carbon numbers refer to the number of carbon atoms that form the ring system. A C6 ring system, i.e., a phenyl ring, is an exemplary aryl group. In various embodiments, the polycyclic ring is a bicyclic aryl group, where exemplary bicyclic aryl groups are C8-C12, or C9-C10.

“Arylene” is a polyvalent, aromatic hydrocarbon, ring system. The ring system may be monocyclic or fused polycyclic (e.g., bicyclic, tricyclic, etc.). In some embodiments, the monocyclic arylene group is C5-C10, or C5-C7, or C5-C6, where these carbon numbers refer to the number of carbon atoms that form the ring system. A C6 ring system, i.e., a phenylene ring, is an exemplary aryl group. In some embodiments, the polycyclic ring is a bicyclic arylene group, where exemplary bicyclic arylene groups are C8-C12, or C9-C10. The arylene group may be divalent, i.e., it has two open sites that each bond to another group; or trivalent, i.e., it has three open sites that each bond to another group; or it may have more than three open sites.

“Carbocycle” refers to a ring formed exclusively from carbon, which may be saturated or unsaturated, including aromatic. The ring may be monocyclic (e.g., cyclohexyl, phenyl), bicyclic (e.g., norbornyl), polycyclic (e.g., adamantyl) or contain a fused ring system (e.g., decalinyl, naphthyl). In one embodiment, the ring is monocyclic and formed from 5, 6 or 7 carbons. In one embodiment, the ring is bicyclic and formed from 7, 8 or 9 carbons. In one embodiment, the ring is polycyclic and formed from 9, 10 or 11 carbons. In one embodiment, the ring includes a fused ring system and is formed from 8-12 carbons. Thus, in one embodiment, the carbocycle is formed from 5-12 ring carbons.

“Heteroalkyl” is an alkyl group (as defined herein) wherein at least one of the carbon atoms is replaced with a heteroatom. Exemplary heteroatoms are nitrogen, oxygen, sulfur, and halogen. A heteroatom may, but typically does not, have the same number of valence sites as carbon. Accordingly, when a carbon is replaced with a heteroatom, the number of hydrogens bonded to the heteroatom may need to be increased or decreased to match the number of valence sites of the heteroatom. For instance, if carbon (valence of four) is replaced with nitrogen (valence of three), then one of the hydrogens formerly attached to the replaced carbon must be deleted. Likewise, if carbon is replaced with halogen (valence of one), then three (i.e., all) of the hydrogens formerly bonded to the replaced carbon must be deleted. As another example, trifluoromethyl is a heteroalkyl group wherein the three methyl groups of a t-butyl group are replaced by fluorine.

“Heteroalkylene” is an alkylene group (as defined herein) wherein at least one of the carbon atoms is replaced with a heteroatom. Exemplary heteroatoms are nitrogen, oxygen, sulfur, and halogen. A heteroatom may, but typically does not, have the same number of valence sites as carbon. Accordingly, when a carbon is replaced with a heteroatom, the number of hydrogens bonded to the heteroatom may need to be increased or decreased to match the number of valence sites of the heteroatom.

“Heteroaryl” is a monovalent aromatic ring system containing carbon and at least one heteroatom in the ring. The heteroaryl group may, in various embodiments, have one heteroatom, or 1-2 heteroatoms, or 1-3 heteroatoms, or 1-4 heteroatoms in the ring. Heteroaryl rings may be monocyclic or polycyclic, where the polycyclic ring may contain fused, spiro or bridged ring junctions. In one embodiment, the heteroaryl is selected from monocyclic and bicyclic. Monocyclic heteroaryl rings may contain from about 5 to about 10 member atoms (carbon and heteroatoms), e.g., from 5-7, and most often from 5-6 member atoms in the ring. Bicyclic heteroaryl rings may contain from about 8-12 member atoms, or 9-10 member atoms in the ring. The heteroaryl ring may be unsubstituted or substituted. In one embodiment, the heteroaryl ring is unsubstituted. In another embodiment, the heteroaryl ring is substituted. Exemplary heteroaryl groups include benzofuran, benzothiophene, furan, imidazole, indole, isothiazole, oxazole, piperazine, pyrazine, pyrazole, pyridazine, pyridine, pyrimidine, pyrrole, quinoline, thiazole and thiophene.

“Heteroarylene” is a polyvalent aromatic ring system containing carbon and at least one heteroatom in the ring. In other words, a heteroarylene group is a heteroaryl group that has more than one open site for bonding to other groups. The heteroarylene group may, in various embodiments, have one heteroatom, or 1-2 heteroatoms, or 1-3 heteroatoms, or 1-4 heteroatoms in the ring. Heteroarylene rings may be monocyclic or polycyclic, where the polycyclic ring may contain fused, spiro or bridged ring junctions. In one embodiment, the heteroaryl is selected from monocyclic and bicyclic. Monocyclic heteroarylene rings may contain from about 5 to about 10 member atoms (carbon and heteroatoms), e.g., from 5-7, or from 5-6 member atoms in the ring. Bicyclic heteroarylene rings may contain from about 8-12 member atoms, or 9-10 member atoms in the ring.

“Heteroatom” is a halogen, nitrogen, oxygen, silicon or sulfur atom. Groups containing more than one heteroatom may contain different heteroatoms.

“Heterocycle” refers to a ring containing at least one carbon and at least one heteroatom. The ring may be monocyclic (e.g., morpholinyl, pyridyl), bicyclic (e.g., bicyclo[2.2.2]octyl with a nitrogen at one bridgehead position), polycyclic, or contain a fused ring system. In one embodiment, the ring is monocyclic and formed from 5, 6 or 7 atoms. In one embodiment, the ring is bicyclic and formed from 7, 8 or 9 atoms. In one embodiment, the ring is polycyclic and formed from 9, 10 or 11 atoms. In one embodiment, the ring includes a fused ring system and is formed from 8-12 atoms. Thus, in one embodiment, the heterocycle is formed from 5-12 ring atoms. In one embodiment, the heteroatom is selected from oxygen, nitrogen and sulfur. In one embodiment, the heterocycle contains 1, 2 or 3 heteroatoms.

“Pharmaceutically acceptable salt” and “salts thereof” in the compounds of the present invention refers to acid addition salts and base addition salts.

Acid addition salts refer to those salts formed from compounds of the present invention and inorganic acids such as hydrochloric acid, hydrobromic acid, sulfuric acid, nitric acid, phosphoric acid and the like, and/or organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

Base addition salts refer to those salts formed from compounds of the present invention and inorganic bases such as sodium, potassium, lithium, ammonium, calcium, magnesium, iron, zinc, copper, manganese, aluminum salts and the like. Suitable salts include the ammonium, potassium, sodium, calcium and magnesium salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, trimethamine, dicyclohexylamine, lysine, arginine, histidine, caffeine, procaines, hydrabamine, choline, betaine, ethylenediamine, glucosamine, methylglucamine, theobromine, purines, piperazine, piperidine, N-ethylpiperidine, and the like.

As used herein, the terms “treatmen,t” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

The terms “individual,” “host,” “subject,” and “patient,” used interchangeably herein, refer to a mammal, e.g., a human, or a non-human mammal, including, e.g., equines, murines (rats, mice), bovines, ovines, felines, canines, non-human primates, etc.

The terms “cancer,” “neoplasm,” and “tumor” are used interchangeably herein to refer to cells which exhibit relatively autonomous growth, so that they exhibit an aberrant growth phenotype characterized by a significant loss of control of cell proliferation. Cancerous cells can be benign or malignant.

The term “proliferative disorder” and “proliferative disease” are used interchangeably to refer to any disease or condition characterized by pathological or undesired cell growth or proliferation, including disorders resulting from and/or characterized by unrestrained or undesired proliferation of epithelial cells (e.g., fibrotic disorders such as liver fibrosis, renal fibrosis, lung fibrotic disorders, pulmonary fibrosis, idiopathic pulmonary fibrosis, etc.); disorders resulting from and/or characterized by unrestrained or undesired endothelial cells (e.g., angiogenic disorders, such as chronic inflammation); neoplastic disorders (e.g., cancer); and the like.

The term “chemotherapeutic agent” or “chemotherapeutic” (or “chemotherapy”, in the case of treatment with a chemotherapeutic agent) is meant to encompass any non-proteinaceous (i.e., non-peptidic) chemical compound useful in the treatment of cancer. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g. calicheamicin, especially calicheamicin gamma1I and calicheamicin phiI1, see, e.g., Agnew, Chem. Intl. Ed. Engl., 33: 183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubincin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g. paclitaxel (TAXOL®, Bristol Meyers Squibb Oncology, Princeton, N.J.) and docetaxel (TAXOTERE®, Rhone-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine™); novantrone; teniposide; edatrexate; daunomycin; aminopterin; xeoloda; ibandronate; CPT-11; topoisomerase inhibitor RFS 2000; difluromethylomithine (DMFO); retinoids such as retinoic acid; capecitabine; and pharmaceutically acceptable salts, acids or derivatives of any of the above. Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); inhibitors of the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprolide, and goserelin; and pharmaceutically acceptable salts, acids or derivatives of any of the above.

The term “antineoplastic” agent, drug or compound is meant to refer to any agent, including any chemotherapeutic agent, biological response modifier (including without limitation (i) proteinaceous, i.e. peptidic, molecules capable of elaborating or altering biological responses and (ii) non-proteinaceous, i.e. non-peptidic, molecules capable of elaborating or altering biological responses), cytotoxic agent, or cytostatic agent, that reduces proliferation of a neoplastic cell.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a polypeptide N-acetyl-α-galactosaminyltransferase inhibitor” includes a plurality of such inhibitors and reference to “the active agent” includes reference to one or more active agents and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides inhibitors of mucin-type O-linked glycosylation, and in particular inhibitors of polypeptide N-acetyl-α-galactosaminyltransferases; as well as compositions, including pharmaceutical compositions, comprising the inhibitors. The present invention further provides methods of identifying inhibitors of polypeptide N-acetyl-α-galactosaminyltransferases. The present invention further provides various applications that use the inhibitors, including research applications, and treatment methods.

Inhibitors of Polypeptide N-acetyl-alpha-galactosaminyltransferases

The present invention provides inhibitors of mucin-type O-linked glycosylation, and in particular inhibitors of polypeptide N-acetyl-α-galactosaminyltransferases (ppGalNAcTs). The present invention provides compositions, including pharmaceutical compositions, comprising a subject ppGalNAcT inhibitor.

In some embodiments, a subject ppGalNAcT inhibitor inhibits mucin-type O-linked glycosylation in a eukaryotic cell. For example, a subject ppGalNAcT inhibitor inhibits mucin-type O-linked glycosylation in a eukaryotic cell by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, or more, when compared to the level of mucin-type O-linked glycosylation in the cell in the absence of the inhibitor.

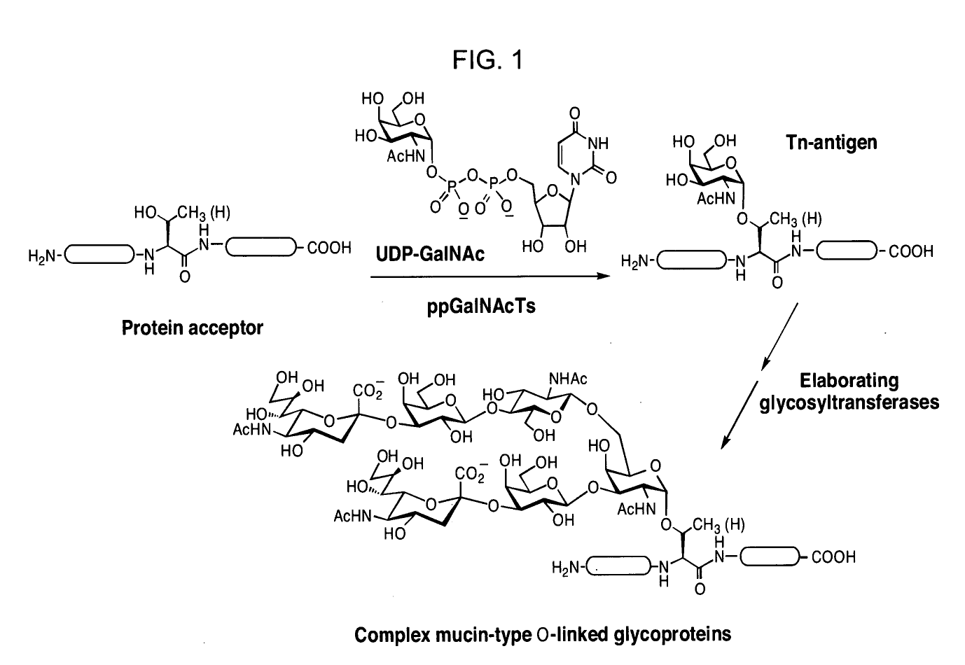

Whether a subject inhibitor inhibits mucin-type O-linked glycosylation in a eukaryotic cell can be readily determined. For example, an assay in which the presence of Tn antigen on the cell surface of a eukaryotic cell can be used. As shown in FIG. 1, Tn antigen is a core glycopeptide comprised of a polypeptide to which is linked a GalNAc molecule. Expression of a Tn antigen on the surface of a eukaryotic cell is readily detectable using an assay that detects binding of a detectably labeled moiety that specifically binds to the Tn antigen, or the GalNAc component of the Tn antigen. Suitable assays include, e.g., use of a detectably labeled antibody that binds specifically to the GalNAc moiety of the Tn antigen; use of a detectably labeled Helix pomatia agglutinin (HPA); and the like. The antibody or the HPA can be directly or indirectly labeled with various labels, including, fluorescent labels; radiolabels; enzyme labels (e.g., where the enzyme gives rise to a detectable product); members of specific binding pairs (e.g., where specific binding pairs include biotin/avidin, lectin/sugars, antibody/antigen, antibody/hapten, etc.). Fluorescent labels are conveniently detected using fluorescence activated cell sorting (FACS) analysis.

In many embodiments, a subject ppGalNAcT inhibitor selectively inhibits mucin-type O-linked glycosylation in a eukaryotic cell, e.g., a subject ppGalNAcT inhibitor inhibits mucin-type O-linked glycosylation in a eukaryotic cell, but does not substantially inhibit N-linked glycosylation, and does not substantially inhibit any O-linked glycosylation other than mucin-type O-linked glycosylation in a eukaryotic cell. Thus, e.g., a subject ppGalNAcT inhibitor inhibits N-linked glycosylation by less than about 10%, less than about 5%, or less than about 2%, and in many embodiments does not detectably inhibit N-linked glycosylation in a eukaryotic cell. Whether a subject ppGalNAcT inhibitor inhibits N-linked glycosylation in a eukaryotic cell is readily determined by analyzing the extent of ConA binding to a eukaryotic cell, e.g., as described in the Example. As a further example, a subject ppGalNAcT inhibitor inhibits O-linked glycosylation other than mucin-type O-linked glycosylation by less than about 10%, less than about 5%, or less than about 2%, and in many embodiments does not detectably inhibit O-linked glycosylation other than mucin-type O-linked glycosylation.

In many embodiments, a subject ppGalNAcT inhibitor selectively inhibits a ppGalNAcT enzyme, e.g., a subject ppGalNAcT inhibitor inhibits a ppGalNAcT enzyme, but does not substantially inhibit a non-ppGalNAcT enzyme. For example, a subject ppGalNAcT inhibitor does not substantially inhibit an enzyme that catalyzes N-linked glycosylation. e.g., a UDP-N-acetylglucosamine-1-P transferase enzyme. UDP-N-acetylglucosamine-1-P transferase enzymes are known in the art. See, e.g., GenBank Accession Nos. NP_{—}001373 and X9H3H5. Thus, in many embodiments, a subject ppGalNAcT inhibitor inhibits an enzyme that catalyzes N-linked glycosylation by less than about 10%, less than about 5%, or less than about 2%, and in many embodiments does not detectably inhibit an enzyme that catalyzes N-linked glycosylation.

As another example, a subject ppGalNAcT inhibitor does not substantially inhibit UDP-sugar-utilizing enzymes other than a ppGalNAcT enzyme. For example, a subject ppGalNAcT inhibitor does not substantially inhibit a β1-4 galactosyltransferase, or an α1-3 galactosyltransferase. Thus, in many embodiments, a subject ppGalNAcT inhibitor inhibits a β1-4 galactosyltransferase or an α1-3 galactosyltransferase by less than about 10%, less than about 5%, or less than about 2%, and in many embodiment does not detectably inhibit a β1-4 galactosyltransferase or an α1-3 galactosyltransferase.

The term “selective inhibitor of ppGalNAcT” is used herein to refer to a compound that selectively inhibits ppGalNAcT activity in preference to an enzyme that catalyzes N-linked glycosylation. e.g., a UDP-N-acetylglucosamine-1-P transferase enzyme (or any other enzyme, e.g., a β1-4 galactosyltransferase or an α1-3 galactosyltransferase) and particularly a compound for which the ratio of the IC₅₀ concentration (concentration inhibiting 50% of activity) for ppGalNAcT to the IC₅₀ concentration of the same compound for, e.g., UDP-N-acetylglucosamine-1-P transferase, is greater than 1. Such ratio is readily determined by assaying for ppGalNAcT activity and assaying for UDP-N-acetylglucosamine-1-P transferase activity in the presence of the compound and from the resulting data obtaining a ratio of IC₅₀s.

In some embodiments, a subject ppGalNAcT inhibitor inhibits any ppGalNAcT enzyme. In other embodiments, a subject ppGalNAcT inhibitor inhibits a subset of ppGalNAcT enzymes. For example, in some embodiments, a subject ppGalNAcT inhibitor inhibits 2, 3, 4, or 5 ppGalNAcT enzymes from a given species, and does not substantially inhibit other ppGalNAcT enzymes from the same species. In still other embodiments, a subject ppGalNAcT inhibitor inhibits only one ppGalNAcT enzyme of a given species, and orthologs in other species. In still other embodiments, a subject ppGalNAcT inhibitor inhibits only one ppGalNAcT enzyme of a given species, and does not substantially inhibit orthologous ppGalNAcT enzymes from other species.

In some embodiments, a subject ppGalNAcT inhibitor inhibits a ppGalNAcT enzyme in a tissue-specific and/or cell type-specific manner. Thus, e.g., in some embodiments, a subject ppGalNAcT inhibitor inhibits a liver-specific ppGalNAcT enzyme, and does not substantially inhibit a ppGalNAcT enzyme from an organ other than the liver. As another non-limiting example, in some embodiments a subject ppGalNAcT inhibitor inhibits an endothelial cell-specific ppGalNAcT, and does not substantially inhibit a ppGalNAcT present in a non-endothelial cell. As another non-limiting example, in some embodiments a subject ppGalNAcT inhibitor inhibits a gut epithelial cell-specific ppGalNAcT, and does not substantially inhibit a ppGalNAcT present in a cell other than a gut epithelial cell.

In some embodiments, a subject ppGalNAcT inhibitor inhibits enzymatic activity of a ppGalNAcT enzyme with an IC₅₀ of less than about 100 μM, e.g., a subject ppGalNAcT inhibitor inhibits a ppGalNAcT enzyme with an IC₅₀ of less than about 100 μM, less than about 75 μM, less than about 50 μM, less than about 40 μM, less than about 25 μM, less than about 10 μM, less than about 1 μM, less than about 100 nM, less than about 80 nM, less than about 60 nM, less than about 50 nM, less than about 25 nM, less than about 10 nM, or less than about 1 nM, or less. Thus, in some embodiments, a subject ppGalNAcT inhibitor inhibits a ppGalNAcT enzyme with an IC₅₀ of from about 100 μM to about 75 μM, from about 75 μM to about 50 μM, from about 50 μM to about 40 μM, from about 40 μM to about 30 μM, from about 30 μM to about 20 μM, from about 20 μM to about 10 μM, from about 10 μM to about 1 μM, from about 1 μM to about 100 nM, from about 100 nM to about 10 nM, or from about 10 nM to about 1 nM. In some embodiments, a subject ppGalNAcT inhibitor inhibits a ppGalNAcT enzyme with an IC₅₀ of from about 5 μM to about 50 μM, or from about 5 μM to about 40 μM.

In some embodiments, a subject ppGalNAcT inhibitor induces apoptosis in a eukaryotic cell. In some embodiments, an “effective amount” of a ppGalNAcT inhibitor is an amount that induces apoptosis in a cell.

Whether apoptosis is induced in a eukaryotic cell is readily determined using any known method. Assays can be conducted on cell populations or an individual cell, and include morphological assays and biochemical assays. A non-limiting example of a method of determining the level of apoptosis in a cell or a cell population is TUNEL (TdT-mediated dUTP nick-end labeling) labeling of the 3′-OH free end of DNA fragments produced during apoptosis (Gavrieli et al. (1992) J. Cell Biol. 119:493). The TUNEL method involves catalytically adding a nucleotide, which has been conjugated to a chromogen system or a to a fluorescent tag, to the 3′-OH end of the 180-bp (base pair) oligomer DNA fragments in order to detect the fragments. The presence of a DNA ladder of 180-bp oligomers is indicative of apoptosis. Procedures to detect cell death based on the TUNEL method are available commercially, e.g., from Boehringer Mannheim (Cell Death Kit) and Oncor (Apoptag Plus). Another marker that is currently available is annexin, sold under the trademark APOPTEST™. This marker is used in the “Apoptosis Detection Kit,” which is also commercially available, e.g., from R&D Systems. During apoptosis, a cell membrane's phospholipid asymmetry changes such that the phospholipids are exposed on the outer membrane. Annexins are a homologous group of proteins that bind phospholipids in the presence of calcium. A second reagent, propidium iodide (PI), is a DNA binding fluorochrome. When a cell population is exposed to both reagents, apoptotic cells stain positive for annexin and negative for PI, necrotic cells stain positive for both, live cells stain negative for both. Other methods of testing for apoptosis are known in the art and can be used, including, e.g., the method disclosed in U.S. Pat. No. 6,048,703.

ppGalNAcT

Polypeptide N-acetyl-α-galactosaminyltransferases are known in the art. These enzymes are also referred to in the art as UDP-GalNAc:polypeptide N-acetylgalactosaminyltransferases. See, e.g., Ten Hagen et al. ((2003) Glycobiology 13:1-16). As used herein, the term “ppGalNAcT” includes any of the known ppGalNAcT enzymes. For example, there are several known human ppGalNAcT enzymes. See, e.g., GenBank Accession Nos. NP_{—}065207 (human ppGalNAcT-1); NP_{—}004472 (human ppGalNAcT-2); NP_{—}004473 (human ppGalNAcT-3); NP_{—}003765 (human ppGalNAcT-4); NP_{—}055383 (human ppGalNAcT-5); NP_{—}009141 (human ppGalNAcT-6); NP_{—}473451 (human ppGalNAcT-7); NP_{—}059113 (human ppGalNAcT-8); NP_{—}065580 (human ppGalNAcT-9); NP_{—}938080 (human ppGalNAcT-10); NP_{—}071370 (human ppGalNAcT-11); NP_{—}078918 (human ppGalNAcT-12); XP_{—}054951 (human ppGalNAcT-13); NP_{—}078848 (human ppGalNAcT-14); and NP_{—}660335 (human ppGalNAcT-15). In addition, there are several known ppGalNAcT enzymes from mouse, rat, cow, insect (e.g., Drosophila melanogaster), cryptosporidium, Toxoplasma gondii, nematodes, etc., the sequences of which can be found on the internet at ncbi.nlm.nih.gov/entrez. See also Ten Hagen et al. (2003) Glycobiology 13:1R-16R.

In some embodiments, a ppGalNAcT comprises an amino acid sequence as set forth in any of the aforementioned GenBank Accession numbers, or any counterpart of such ppGalNAcT in another species, e.g., in another mammalian species, in another vertebrate species, in another eukaryotic invertebrate species, or in any other eukaryotic species. Thus, the source of the ppGalNAcT can be any eukaryote, including, but not limited to, mammals (including, but not limited to, human, mouse, and rat); reptiles; amphibians; birds; plants; insects; arachnids; invertebrates; yeast; molds; algae; fungi; nematodes; protozoa; helminths; crustaceans; sponges; mollusks; and the like.

In some embodiments, a ppGalNAcT polypeptide is an isolate from a naturally-occurring source of the protein. In other embodiments, a ppGalNAcT polypeptide is a recombinant protein. In other embodiments, a ppGalNAcT polypeptide is a synthetic protein.

The sequence of any known ppGalNAcT polypeptide may be altered in various ways known in the art to generate targeted changes in sequence. A variant polypeptide will usually be substantially similar any known ppGalNAcT amino acid sequence, i.e. will differ by at least one amino acid, and may differ by at least two but generally not more than about ten amino acids. The sequence changes may be substitutions, insertions or deletions. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Typically, a ppGalNAcT polypeptide is enzymatically active, e.g., a ppGalNAcT polypeptide carries out O-linked glycosylation of a peptide or a polypeptide substrate, as shown in FIG. 1. Those skilled in the art are aware of changes that can be made to a ppGalNAcT amino acid sequence without altering substantially the enzymatic activity of the polypeptide. For example, mutagenesis of murine ppGalNAcT-T1 to create D156Q, D209N, H211D, E127Q, E213Q, E319Q, E322Q, or D310N substitutions resulted in drastic reduction in enzymatic activity, or undetectable enzymatic activity, while mutations in the C-terminal ricin-like lectin motif did not alter the enzyme's catalytic properties. Hagen et al. (1999) J. Biol. Chem. 274:6797-6803.

The term “ppGalNAcT polypeptide” includes enzymatically active fragments of a ppGalNAcT polypeptide. In general, an enzymatically active fragment is a fragment of a ppGalNAcT of at least about 50 amino acids, at least about 75 amino acids, at least about 100 amino acids, at least about 125 amino acids, at least about 150 amino acids, at least about 200 amino acids, at least about 250 amino acids, at least about 300 amino acids, at least about 350 amino acids, at least about 400 amino acids, at least about 450 amino acids, at least about 500 amino acids, at least about 550 amino acids, or at least about 600 amino acids in length. Whether a given fragment is enzymatically active is readily determined using any known method, or a method as described in the Example.

A ppGalNAcT polypeptide may be a fusion protein, e.g., a protein comprising a ppGalNAcT polypeptide, or enzymatically active fragment thereof, and a non-ppGalNAcT fusion partner, where suitable fusion partners include, but are not limited to, enzymes that produce detectable products (e.g., horse radish peroxidase (HRP), β-galactosidase, luciferase, etc.); antibodies, antibody fragments, immunoglobulins, or fragments of an immunoglobulin (e.g., an immunoglobulin Fc portion, an antigen-binding fragment of an antibody, etc.); epitope tags (e.g., hemagglutinin, flagellin tags, etc.); moieties that provide for facility of purification (e.g., histidine tags, such as (His)ₙ, where n=3-10; glutathione-5-transferase; and the like); fluorescent proteins (e.g., green fluorescent proteins; a fluorescent protein from any Anthozoan species); a chromogenic protein; a luminescent protein; metal-binding proteins and metal-binding fragments thereof; and the like.

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation (modification with a polyethylene glycol moiety or moieties); and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

ppGalNAcTs initiate mucin-type O-linked glycosylation in the Golgi apparatus by catalyzing the transfer of GalNAc to serine and threonine residues on target proteins. They may be characterized by having one or more of the following: an approximately 112-amino acid glycosyltransferase 1 motif representing the first half of the catalytic unit and containing a short aspartate-any residue-histidine (DXH) or aspartate-any residue-aspartate (DXD)-like sequence; a second half of the catalytic unit containing a DXXXXXWGGENXE motif (where X=any amino acid); and an approximately 128-amino acid C-terminal ricin-like lectin motif containing domain.

A ppGalNAcT polypeptide can be produced by any known method. DNA sequences encoding a ppGalNAcT polypeptide may be synthesized using standard methods. In many embodiments, a ppGalNAcT polypeptide is the product of expression of manufactured DNA sequences (e.g., recombinant nucleic acids; synthetic nucleic acids) transformed or transfected into bacterial hosts, e.g., E. coli, or in eukaryotic host cells (e.g., yeast; mammalian cells, such as CHO cells; and the like). In these embodiments, the ppGalNAcT is “recombinant ppGalNAcT.” Alternatively, a ppGalNAcT polypeptide can be synthesized, using standard methods.

Inhibitors

In some embodiments, a subject ppGalNAcT inhibitor has a structure represented by the generic formula #1 as set forth below.

Generic formula #1:

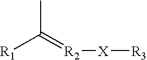

and stereoisomers, solvates, and pharmaceutically acceptable salts thereof, and a pharmaceutically acceptable carrier, diluent or excipient, where each of R₁, R₂, and R₃ is independently selected from alkyl, aryl and heteroaryl, wherein each of alkyl, aryl and heteroaryl may be substituted with one or more groups selected from C₁-C₂₀alkyl, C₆-C₁₀aryl, heteroalkyl and heteroaryl; and where X is a linker of any length or structure. In some embodiments, each of R₁ and R₃ is independently selected from —O—CH₃ and —OH. In some embodiments, R₂ is selected from N, S, and O.

In some embodiments, X is selected from O, (CH₂)ₙ, where n is an integer from 1 to 10, an amido group, 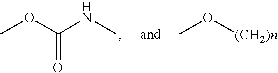
where n is an integer from 1 to 10.

In some embodiments, R₁ has the structure:

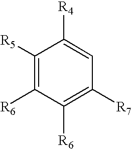

where each of R₄-R₇ is independently H, hydroxyl, aryl, alkyl, cycloalkyl, and —NH₂. In some particular embodiments, R₁ has the structure:

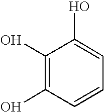

In some embodiments, R₃ is uridine or a uridine derivative. In some embodiments, R₃ has the structure:

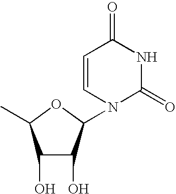

In some embodiments, a subject ppGalNAcT inhibitor is a compound of generic formula #2, as shown below.

Generic formula #2:

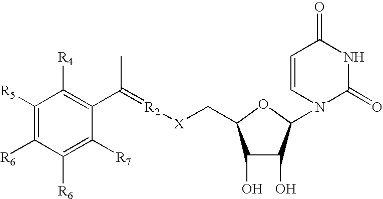

and stereoisomers, solvates, and pharmaceutically acceptable salts thereof, and a pharmaceutically acceptable carrier, diluent or excipient, wherein R₂ is selected from N, S, and O; wherein each of R₄ through R₇ is independently H, hydroxyl, aryl, alkyl, cycloalkyl, and —NH₂ and wherein X is X is selected from O, (CH₂)ₙ, where n is an integer from 1 to 10, an amido group, 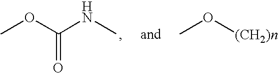
where n is an integer from 1 to 10.

In some embodiments, a subject ppGalNAcT inhibitor is a compound of generic formula #3, as shown below.

Generic formula #3:

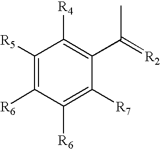

and stereoisomers, solvates, and pharmaceutically acceptable salts thereof, and a pharmaceutically acceptable carrier, diluent or excipient, wherein R₂ is selected from N, S, and O; and wherein each of R₄ through R₇ is independently H, hydroxyl, aryl, alkyl, cycloalkyl, and —NH₂.

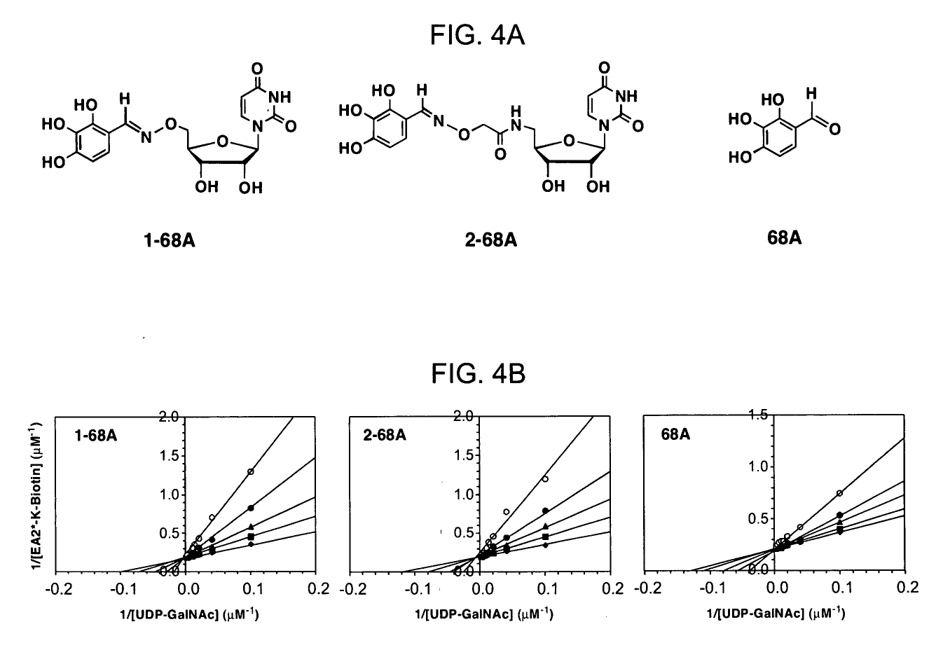

In particular embodiments, a subject inhibitor is a compound having any one of the structures depicted in FIG. 4A and described in the Example, and stereoisomers, solvates, and pharmaceutically acceptable salts thereof. In some embodiments, a subject composition comprises a compound having any one of the structures depicted in FIG. 4A and described in the Example, and stereoisomers, solvates, and pharmaceutically acceptable salts thereof, and a pharmaceutically acceptable carrier, diluent or excipient.

A subject inhibitor is not a UDPGlcNAc 4-epimerase inhibitor, e.g., as depicted in Winans and Bertozzi (2002) Chemistry and Biology 9:113-129. Furthermore, a subject inhibitor is not a UDP-galactopyranose inhibitor, e.g., as described in Scherman et al. (2003) Antimicrob. Agents Chemother. 47:378-382.

Inhibitor Compositions

The present invention further provides compositions comprising a subject inhibitor. A subject composition comprises a subject inhibitor, and may further comprise one or more additional components, such as a buffer, a vehicle, an adjuvant, a carrier, a diluent, a pH adjusting agent (e.g., a buffer such as a Tris buffer, a phosphate buffer, etc.), a solubilizing agent, an ionic detergent, a non-ionic detergent, a salt (e.g., NaCl, MgCl₂, MgSO₄, KCl, and the like), a tonicity adjusting agent, a stabilizer, a wetting agent, a preservative, and the like. In some embodiments, a subject composition comprises a subject inhibitor and a pharmaceutically acceptable excipient. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, a subject inhibitor (also referred to herein as “an active agent,” “a compound,” “an agent,” and similar terms) is prepared in a pharmaceutically acceptable composition for delivery to a host.

Pharmaceutically acceptable carriers preferred for use with a subject agent may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, and microparticles, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. A composition comprising a subject agent may also be lyophilized using means well known in the art, for subsequent reconstitution and use according to the invention.

For oral preparations, the agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent (e.g., a subject inhibitor) can be formulated into preparations for injection by dissolving, suspending or emulsifying the agent in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. An active agent (e.g., a subject inhibitor) can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent calculated in an amount sufficient to produce the desired effect in association with a pharmaceutically acceptable diluent, carrier or vehicle. The specifications for the unit dosage forms of the present invention depend on the particular compound employed and the effect to be achieved, and the pharmacodynamics associated with each compound in the host.

A subject formulation comprising an active agent in some embodiments includes one or more of an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylprrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Tablets comprising an active agent may be coated with a suitable film-forming agent, e.g., hydroxypropylmethyl cellulose, hydroxypropyl cellulose or ethyl cellulose, to which a suitable excipient may optionally be added, e.g., a softener such as glycerol, propylene glycol, diethylphthalate, or glycerol triacetate; a filler such as sucrose, sorbitol, xylitol, glucose, or lactose; a colorant such as titanium hydroxide; and the like.

In general, the pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions comprising the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. Preservatives and other additives may also be present such as, for example, antimicrobials, antioxidants, chelating agents, and inert gases and the like. In one embodiment, a subject agent formulation comprises additional agents, e.g., an anti-mycobacterial agent, an anti-bacterial agent(s), a tumoricidal agent, etc.

A subject agent can be administered in the absence of agents or compounds that might facilitate uptake by target cells. A subject agent can be administered with compounds that facilitate uptake of a subject agent by target cells (e.g., by macrophages) or otherwise enhance transport of a subject agent to a treatment site for action. Absorption promoters, detergents and chemical irritants (e.g., keratinolytic agents) can enhance transmission of a subject agent into a target tissue (e.g., through the skin). For general principles regarding absorption promoters and detergents which have been used with success in mucosal delivery of organic and peptide-based drugs, see, e.g., Chien, Novel Drug Delivery Systems, Ch. 4 (Marcel Dekker, 1992). Suitable agents for use in the method of this invention for mucosal/nasal delivery are also described in Chang, et al., Nasal Drug Delivery, “Treatise on Controlled Drug Delivery”, Ch. 9 and Tables 3-4B thereof, (Marcel Dekker, 1992). Suitable agents which are known to enhance absorption of drugs through skin are described in Sloan, Use of Solubility Parameters from Regular Solution Theory to Describe Partitioning-Driven Processes, Ch. 5, “Prodrugs: Topical and Ocular Drug Delivery” (Marcel Dekker, 1992), and at places elsewhere in the text. All of these references are incorporated herein for the sole purpose of illustrating the level of knowledge and skill in the art concerning drug delivery techniques.

A colloidal dispersion system may be used for targeted delivery of the subject agent to specific tissue. Colloidal dispersion systems include macromolecule complexes, nanocapsules, microspheres, beads, and lipid-based systems including oil-in-water emulsions, micelles, mixed micelles, and liposomes.

Liposomes are artificial membrane vesicles which are useful as delivery vehicles in vitro and in vivo. It has been shown that large unilamellar vesicles (LUV), which range in size from 0.2-4.0 Fm can encapsulate a substantial percentage of an aqueous buffer comprising large macromolecules. A subject compound can be encapsulated within the aqueous interior and be delivered to cells in a biologically active form (Fraley, et al., (1981) Trends Biochem. Sci., 6:77). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

Where desired, targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various well known linking groups can be used for joining the lipid chains to the targeting ligand (see, e.g., Yanagawa, et al., (1988) Nuc. Acids Symp. Ser., 19:189; Grabarek, et al., (1990) Anal. Biochem., 185:131; Staros, et al., (1986) Anal. Biochem. 156:220 and Boujrad, et al., (1993) Proc. Natl. Acad. Sci. USA, 90:5728). Targeted delivery of a subject agent can also be achieved by conjugation of a subject agent to the surface of viral and non-viral recombinant expression vectors, to an antigen or other ligand, to a monoclonal antibody or to any molecule which has the desired binding specificity.

Screening Methods

The present invention further provides methods of identifying modulators of ppGalNAcT enzymatic activity. In particular, the present invention provides in vitro cell-free, non-radioactive methods for identifying modulators of ppGalNAcT enzymatic activity. The methods generally involve contacting an enzymatically active ppGalNAcT polypeptide with a ppGalNAcT substrate and a test agent; and determining the effect, if any, of the test agent on ppGalNAcT enzymatic activity. The assay is designed such that the read-out for an effect of the test agent on ppGalNAcT enzymatic activity is a non-radioactive signal.

A test agent that inhibits ppGalNAcT enzyme activity by at least about 10%, at least about 20%, at least about 25%, at least about 30%, at least about 35%, at least about 40%, at least about 50%, at least about 60%, at least about 70%, at least about 80%, or at least about 90%, or more, compared to the enzymatic activity of the ppGalNAcT polypeptide in the absence of the test agent, is an agent that inhibits ppGalNAcT enzymatic activity. Agents that inhibit ppGalNAcT activity at least about 10%, at least about 20%, at least about 25%, at least about 30%, at least about 35%, at least about 40%, at least about 50%, at least about 60%, at least about 70%, at least about 80%, or at least about 90%, or more, compared to the enzymatic activity of the ppGalNAcT polypeptide in the absence of the agent, are candidate agents for use in a subject research application or therapeutic application.

Of particular interest are agents that selectively inhibit enzymatic activity of a ppGalNAcT. Identification of an agent that selectively inhibits a ppGalNAcT can be performed by determining the effect of the test agent on an enzyme that catalyzes N-linked glycosylation; and/or determining the effect of the test agent on a UDP sugar-utilizing enzyme other than a ppGalNAcT, e.g., β1-4GalT or α1-3GalT. Agents that inhibit enzymatic activity of a ppGalNAcT, but do not substantially inhibit the enzymatic activity of an enzyme that catalyzes N-linked glycosylation, and/or a UDP sugar-utilizing enzyme other than a ppGalNAcT, are selective ppGalNAcT inhibitors.

Of particular interest in some embodiments are methods of identifying agents that inhibit the enzymatic activity of a subset of ppGalNAcT enzymes, or a specific ppGalNAcT. Such agents can be identified by screening for test agents that inhibit a given ppGalNAcT; and counter-testing for an effect of the test agent on inhibition of a different ppGalNAcT.

In general, a subject screening method involves: a) contacting a ppGalNAcT polypeptide with a peptide substrate and a test agent; and b) determining the effect, if any, of the test agent on N-acetyl galactosamine (GalNAc) modification of the peptide substrate by the ppGalNAcT polypeptide. The determining generally involves detecting binding of a detectably labeled moiety that binds the GalNAc-modified peptide substrate. The detectable label is a non-radioactive label.

The terms “candidate agent,” “agent,” “substance,” “test agent,” and “compound” are used interchangeably herein. Test agents encompass numerous chemical classes, and are generally synthetic, semi-synthetic, or naturally occurring inorganic or organic molecules. Candidate agents may be small organic compounds having a molecular weight of more than 50 and less than about 10,000 daltons, e.g., the test agents are generally in the molecular weight range of from about 50 daltons to about 100 daltons, from about 100 daltons to about 200 daltons, from about 200 daltons to about 500 daltons, from about 500 daltons to about 1000 daltons, from about 1000 daltons to about 2000 daltons, from about 2000 daltons to about 5000 daltons, or from about 5000 daltons to about 10,000 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible. Another suitable library is a uridine-based library discussed in Winans and Bertozzi (2002) Chemistry & Biology 9:113-129.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

A candidate agent is assessed for any cytotoxic activity it may exhibit toward control eukaryotic cells, using well-known assays, such as trypan blue dye exclusion, an MTT ([3-(4,5-dimethylthiazol-2-yl)-2,5-diphenyl-2H-tetrazolium bromide]) assay, and the like. Agents that do not exhibit cytotoxic activity toward control cells are considered suitable candidate agents. However, in some embodiments, cytotoxic activity is a desirable attribute of a candidate agent, e.g., where the screening assay identifies compounds that induce apoptosis in a eukaryotic cell.

A variety of reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, anti-microbial agents, etc. may be used. The components may be added in any order. Incubations are performed at any suitable temperature, typically between 37° C. and 40° C. Incubation periods are selected for optimum activity, but may also be optimized to facilitate rapid high-throughput screening. Typically between 0.1 and 1 hour will be sufficient.

Assays of the invention usually include one or more controls. Thus, a test sample includes a test agent (and typically a peptide substrate and a ppGalNAcT enzyme), and a control sample has all the components of the test sample except for the test agent.

Suitable substrates for a ppGalNAcT polypeptide include any known ppGalNAcT substrate. Suitable substrates include peptides comprising at least one threonine residue and/or at least one serine residue. In some embodiments, a suitable peptide substrate is a mono-, di-, tri-, or tetra-substituted glycopeptide, e.g., a peptide having two or more serine and/or threonine residues, where one or more of the serine and/or threonine residues is glycosylated.

Suitable ppGalNAcT substrates include, but are not limited to, a peptide comprising the amino acid sequence GTTPAPVTTSTTSAP (SEQ ID NO:01; Ten Hagen et al. (1999) J. Biol. Chem. 274(39):27867-74), and a mono-, di-, tri-, or tetra-substituted glycosylated derivative thereof; a peptide comprising the amino acid sequence PPDAATAAPLR (SEQ ID NO:02; Wragg et al. (1995) J. Biol. Chem. 270:16947-16954); a peptide comprising the amino acid sequence QTSSPNTGKTSTISTT (SEQ ID NO:03); a peptide comprising the amino acid sequence CPPTPSATTPAPPSSSAPPETTAA (SEQ ID NO:04); a peptide comprising the amino acid sequence Ac-QATEYEYLDYDFLPETEPPEM (SEQ ID NO:05); a peptide comprising the amino acid sequence Ac-CRIQRGPGRAFVTIGKIGNMR (SEQ ID NO:06); a peptide comprising the amino acid sequence AHGVTSAPDTR (SEQ ID NO:07); a peptide comprising the amino acid sequence AHGVTSAPDTRPAPGSTAPPA (SEQ ID NO:08; Hanisch et al. (1999) J. Biol. Chem. 274:9946-9954); a peptide comprising the amino acid sequence VTPRTPPP (SEQ ID NO:09); a peptide comprising the amino acid sequence PTTTPLK (SEQ ID NO:10; Takeuchi et al. (2002) Eur. J. Biochem. 269:6173); a peptide comprising the amino acid sequence PTTTPITTTTK (SEQ ID NO:11; Kato et al. (2001) Glycobiology 11:821-829); a peptide comprising the amino acid sequence PTTDSTTPAPTTK (SEQ ID NO:12; Albone et al. (1999) J. Biol. Chem. 269:16845-16852); a peptide comprising the amino acid sequence PTTTPISTTTMVTPTPTPTC (SEQ ID NO:13); a peptide comprising the amino acid sequence DSTTPAPTTK (SEQ ID NO:14); and a peptide comprising the amino acid sequence GTTPSPVPTTSTTSAP (SEQ ID NO:15). All peptide sequences are given in the amino terminus to carboxyl terminus orientation; “Ac” is an acetyl group; and underlined residues are glycosylated. Also suitable for use are tandem repeats of any known ppGalNAcT peptide substrate. Also suitable for use are mono-, di-, tri-, or tetra-substituted glycosylated derivatives of any of the foregoing peptide substrates. In addition to the above-mentioned peptide substrates, any of the peptide substrates known and described in the art can be used. See, e.g., the peptide substrates discussed in Hanisch et al. (2001) Glycobiology 11:731-740; and Ten Hagen et al. (2003) Glycobiology 13: 1R-16R.

A peptide substrate may further comprise a moiety (an “immobilization moiety”) that provides for immobilization of the peptide substrate. Suitable moieties include epitope tags (e.g., for binding to an immobilized antibody specific for the epitope); poly-histidine tracts (e.g., for binding to an immobilized metal ion); a member of a specific binding pair, e.g., biotin; antibodies or antigen-binding fragments thereof; immunoglobulins or immunoglobulin fragments, e.g., Fc portion; and the like. The immobilization moiety can be linked to the carboxyl terminus or the amino terminus of the peptide substrate.

The peptide substrate is typically immobilized, directly or indirectly, on an insoluble support, e.g., a bead, a magnetic bead, a plastic surface, such as a well of a multi-well plate, etc. Any means of immobilizing the peptide substrate can be used. For example, an antibody specific for the peptide is immobilized on an insoluble support, and the peptide substrate is bound to the immobilized antibody, thereby immobilizing the peptide substrate. As another example, a peptide substrate comprises an epitope tag, and the peptide substrate is immobilized by binding to an immobilized antibody specific for the epitope tag. As another example, a peptide substrate comprises a metal ion binding moiety, and the peptide substrate is immobilized by binding of the metal ion binding moiety to an immobilized metal ion. As another example, a peptide substrate comprises a biotin molecule conjugated to the peptide substrate, and the peptide substrate is immobilized by binding of the biotin to immobilized avidin.

The peptide substrate is contacted with a ppGalNAcT enzyme and a test agent; and the effect, if any, of the test agent on N-acetyl galactosamine (GalNAc) modification of the peptide substrate by the ppGalNAcT polypeptide is determined. The determining step generally involves detecting binding of a detectably labeled moiety that binds GalNAc-modified peptide substrate.

In some embodiments, the detectably labeled moiety that binds the GalNAc-modified peptide substrate is a detectably labeled lectin that has affinity for terminal α-N-acetyl-D-galactosaminyl residues. Suitable lectins include, but are not limited to Helix pomatia agglutinin (HPA); Helix aspersa agglutinin (HAA); and the like; and binding fragments thereof. In some embodiments, the detectably labeled moiety is HPA, or a terminal α-N-acetyl-D-galactosamine-binding fragment thereof.

In other embodiments, the detectably labeled moiety that binds GalNAc-modified peptide substrate is a detectably labeled antibody that specifically binds the GalNAc-modified peptide substrate (but not unmodified peptide substrate). The antibody can be a whole antibody, or an antigen-binding fragment, e.g., Fab fragment, an Fv fragment, etc.

The moiety that binds GalNAc-modified peptide substrate is detectably labeled with a label other than a radioactive label. Suitable detectable labels include, but are not limited to, fluorescent proteins; enzymes that give rise to detectable products, e.g., β-galactosidase, alkaline phosphatase, horse radish peroxidase, luciferase, and the like; chromogenic proteins; fluorescent dyes, e.g., coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum dye and the like.

Fluorescent proteins include, but are not limited to, a green fluorescent protein (GFP), including, but not limited to, a GFP derived from Aequoria victoria or a derivative thereof; a GFP from a species such as Renilla reniformis, Renilla mulleri, or Ptilosarcus guernyi, as described in, e.g., WO 99/49019 and Peelle et al. (2001) J Protein Chem. 20:507-519; and any of a variety of fluorescent and colored proteins from Anthozoan species, as described in, e.g., Matz et al. (1999) Nature Biotechnol. 17:969-973; and the like.

Where the detectable label is an enzyme that yields a detectable product, the product can be detected using an appropriate means, e.g., β-galactosidase can, depending on the substrate, yield colored product, which is detected spectrophotometrically, or a fluorescent product; luciferase can yield a luminescent product detectable with a luminometer; etc.

Utility

A subject inhibitor finds use in a variety of applications, including research applications and therapeutic methods.

Research Applications

A subject ppGalNAcT inhibitor finds use in research applications, for analyzing O-linked glycosylation. For example, a subject ppGalNAcT inhibitor is useful in functional studies of mucin-type O-linked glycosylation. A subject ppGalNAcT inhibitor is useful for determining the nature of protein glycosylation, e.g., whether a protein contains O-linked glycosylation can be determined using a subject ppGalNAcT inhibitor.

The present invention provides a method of reducing or inhibiting mucin-type O-linked glycosylation in a eukaryotic cell. The method generally involves contacting the cell with a subject compound. A subject compound inhibits mucin-type O-linked glycosylation by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or more, compared to the level of mucin-type O-linked glycosylation in the absence of the compound. In some embodiments, the cell is a eukaryotic cell in in vitro cell culture. In other embodiments, the cell is a eukaryotic cell in a tissue or an organ in vitro. In other embodiments, the cell is a eukaryotic cell in a tissue or an organ in vivo. In some embodiments, the compound induces apoptosis in the cell.

O-linked glycosylation plays an important role in a number of physiological events and processes. Thus, for example, a subject ppGalNAcT inhibitor finds use in analyzing the role of ppGalNAcT enzymes in embryogenesis.

Of particular interest in some embodiments are ppGalNAcT inhibitors that inhibit only one ppGalNAcT enzyme. Selective ppGalNAcT inhibitors that inhibit only one type of ppGalNAcT enzyme are useful for analyzing the function of various ppGalNAcT enzymes during embryogenesis. Selective ppGalNAcT inhibitors that inhibit only one type of ppGalNAcT enzyme are also useful in dissecting the apparent functional redundancy of this family of enzymes. Selective ppGalNAcT inhibitors that inhibit only one type of ppGalNAcT enzyme are also useful for analyzing the function of ppGalNAcT enzymes that display tissue specificity or restricted distribution of expression across various tissues.

O-glycans serve as the recognition site of many ligands, including lectins. Thus, O-glycyosylation is important in lymphocyte homing, which depends on the interactions between lectins displayed on the surface of certain lymphocytes, and lectin ligands displayed on the surface of certain endothelial cells. A subject ppGalNAcT inhibitor finds use in investigating the role of O-glycosylation on lymphocyte homing, inflammation, and other related processes.

The function of many proteins depends, at least in part, on O-glycosylation. For example, O-glycosylation is important for low-density lipoprotein receptor function and binding. A subject inhibitor allows structure/function analysis of proteins that require O-glycosylation for their function.

Certain cell-cell interactions rely, at least in part, upon the presence of O-glycosylated polypeptides displayed on the cell surface. A subject ppGalNAcT inhibitor is useful in dissecting the role of O-linked glycosylation on cell-cell interactions. Cell-cell interactions of interest include, but are not limited to, lymphocyte-endothelia cell interactions; host-pathogen cell interactions; cell-cell interactions that occur during embryogenesis; cell-cell interactions that occur in the context of cell, tissue, and organ transplantation, e.g., host-donor cell interactions; cell-cell interactions that occur during organogenesis; etc.

A subject ppGalNAcT inhibitor finds use in analyzing the role of ppGalNAcT in tumorigenesis and metastasis. A variety of animal models of various types of neoplasms (including metastatic tumors) can be used to assess the role of ppGalNAcT in tumorigenesis and metastasis.

Biological systems that can be used for analysis of the effect of a given ppGalNAcT inhibitor include cells in in vitro cell culture; and tissues in in vitro culture. Biological systems that can be used for analysis of the effect of a give ppGalNAcT include whole organisms (e.g., in vivo systems). Suitable eukaryotic cells include any eukaryotic cell that synthesizes one or more functional ppGalNAcT enzymes. Whole organisms that are suitable for use in such studies include standard laboratory test organisms such as Drosophila, Caenorhabditis elegans, Arabidopsis, Danio rerio, mice, rats, and the like. Suitable eukaryotic cells include eukaryotic cells that do not normally synthesize a given ppGalNAcT, but that synthesize the ppGalNAcT following introduction into the cell of a recombinant vector that comprises a nucleotide sequence encoding the ppGalNAcT and that provides for production of the encoded ppGalNAcT in the cell.

Methods of Reducing Undesired Cellular Proliferation

The instant invention provides methods of reducing undesired cellular proliferation. As such, the invention provides methods of treating disorders which feature or result from unwanted cellular proliferation. Such disorders include, but are not limited to, neoplastic disorders, disorders resulting from and/or characterized by unrestrained or undesired proliferation of epithelial cells, and disorders resulting from and/or characterized by unrestrained or undesired proliferation of endothelial cells, e.g., unrestrained or undesired angiogenesis. A subject method for reducing undesired cellular proliferation, for treating cancer, for reducing tumor growth, etc., generally involves administering an effective amount of a ppGalNAcT inhibitor to an individual in need thereof (e.g., an individual having cancer, fibrosis, or other form of undesired cellular proliferation). In some embodiments, a ppGalNAcT inhibitor is administered in combination therapy with at least one additional therapeutic agent.

Dosages

Although the dosage used will vary depending on the clinical goals to be achieved, a suitable dosage range of an active agent (e.g., a ppGalNAcT inhibitor) is one which provides from about 1 μg to about 100 mg, e.g., from about 1 μg to about 10 μg, from about 10 μg to about 50 μg, from about 50 μg to about 100 μg, from about 100 μg to about 500 μg, from about 500 μg to about 1 mg, from about 1 mg to about 10 mg, from about 10 mg to about 20 mg, from about 20 mg to about 30 mg, from about 30 mg to about 40 mg, from about 40 mg to about 50 mg, from about 50 mg to about 60 mg, from about 60 mg to about 70 mg, from about 70 mg to about 80 mg, from about 80 mg to about 90 mg, or from about 90 mg to about 100 mg, of an active agent (e.g., a ppGalNAcT inhibitor), administered in a single dose. Alternatively, a target dosage of an active agent (e.g., a ppGalNAcT inhibitor) can be considered to be about in the range of about 0.1-1000 μM, about 0.5-500 μM, about 1-100 μM, or about 5-50 μM in a sample of host blood drawn within the first 24-48 hours after administration of the agent.

Depending on the subject and condition being treated and on the administration route, the subject compounds may be administered in dosages of, for example, 0.1 μg to 10 mg/kg body weight per day. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. Thus, for example, oral dosages may be about ten times the injection dose. Higher doses may be used for localized routes of delivery.

A typical dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

Although the dosage used will vary depending on the clinical goals to be achieved, a suitable dosage range is one which provides up to about 1 μg to about 1,000 μg or about 10,000 μg of a ppGalNAcT inhibitor to reduce a symptom in an individual being treated.

Those of skill will readily appreciate that dose levels can vary as a function of the specific compound, the severity of the symptoms, the pharmacokinetic characteristics of the agent, and the susceptibility of the subject to side effects. Preferred dosages for a given compound are readily determinable by those of skill in the art by a variety of means.

In some embodiments, a single dose of an active agent is administered. In other embodiments, multiple doses of an active agent are administered. Where multiple doses are administered over a period of time, an active agent is administered twice daily (qid), daily (qd), every other day (qod), every third day, three times per week (tiw), or twice per week (biw) over a period of time. For example, an active agent is administered qid, qd, qod, tiw, or biw over a period of from one day to about 2 years or more. For example, an active agent is administered at any of the aforementioned frequencies for one week, two weeks, one month, two months, six months, one year, or two years, or more, depending on various factors.

Routes of Administration

An active agent (e.g., a ppGalNAcT inhibitor) is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, intratumoral, peritumoral, transdermal, subcutaneous, intradermal, transdermal, topical application, intravenous, vaginal, nasal, and other parenteral routes of administration. Suitable routes of administration also include oral and rectal routes. Routes of administration may be combined, if desired, or adjusted depending upon the agent and/or the desired effect. The composition can be administered in a single dose or in multiple doses.

An active agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, vaginal, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

Methods of administration of the agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as cancer or fibrosis. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

Kits with unit doses of the active agent, e.g. in oral or injectable doses, are also provided. In such kits, in addition to the containers containing the unit doses will be an informational package insert describing the use and attendant benefits of the drugs in treating pathological condition of interest. Exemplary compounds and unit doses are those described herein above.

Angiogenesis

In some embodiments, a subject method is effective to reduce angiogenesis by at least about 5%, at least about 10%, at least about 20%, at least about 25%, at least about 50%, at least about 75%, at least about 85%, or at least about 90%, when compared to a suitable control. Thus, in these embodiments, “effective amounts” of an active agent are amounts of active agent that alone or in combination with other therapy for an angiogenesis-associated disorder are sufficient to reduce angiogenesis by at least about 5%, at least about 10%, at least about 20%, at least about 25%, at least about 50%, at least about 75%, at least about 85%, or at least about 90%, when compared to a suitable control. In an experimental animal system, a suitable control may be a genetically identical animal not treated with the subject drug therapy. In non-experimental systems, a suitable control may be degree of angiogenesis existing before administering the subject drug therapy. Other suitable controls may be a placebo control.

In some embodiments, a subject method for reducing angiogenesis or treating an angiogenic disorder in an individual involves administering to an individual in need thereof an effective amount of a ppGalNAcT inhibitor, and an effective amount of an endothelin antagonist. Specific examples of endothelin antagonists useful in the present invention include, but are not limited to, atrasentan (ABT-627; Abbott Laboratories), Veletri™ (tezosentan; Actelion Pharmaceuticals, Ltd.), sitaxsentan (ICOS-Texas Biotechnology), enrasentan (GlaxoSmithKline), darusentan (LU135252; Myogen) BMS-207940 (Bristol-Myers Squibb), BMS-193884 (Bristol-Myers Squibb), BMS-182874 (Bristol-Myers Squibb), J-104132 (Banyu Pharmaceutical), VML 588/Ro 61-1790 (Vanguard Medica), T-0115 (Tanabe Seiyaku), TAK-044 (Takeda), BQ-788 (Banyu Pharmaceutical), BQ123, YM-598 (Yamanouchi Pharma), PD 145065 (Parke-Davis), A-127722 (Abbott Laboratories), A-192621 (Abbott Laboratories), A-182086 (Abbott Laboratories), TBC3711 (ICOS-Texas Biotechnology), BSF208075 (Myogen), S-0139 (Shionogi), TBC2576 (Texas Biotechnology), TBC3214 (Texas Biotechnology), PD156707 (Parke-Davis), PD180988 (Parke-Davis), ABT-546 (Abbott Laboratories), ABT-627 (Abbott Laboratories), SB247083 (GlaxoSmithKline), SB 209670 (GlaxoSmithKline); TRACLEER™ (bosentan; manufactured by Actelion Pharmaceuticals, Ltd.); and an endothelin receptor antagonists discussed in the art, e.g., Davenport and Battistini (2002) Clinical Science 103:15-35, Wu-Wong et al. (2002) Clinical Science 103:1075-1115, and Luescher and Barton (2000) Circulation 102:2434-2440.

In some embodiments, a subject method for reducing angiogenesis or treating an angiogenic disorder in an individual involves administering to an individual in need thereof an effective amount of a ppGalNAcT inhibitor, and an effective amount of a vascular endothelial growth factor (VEGF) antagonist, e.g., a soluble VEGF receptor; an anti-VEGF antibody; an anti-VEGF-receptor (anti-VEGFR) antibody; and the like.

Exemplary non-limiting VEGF antagonists that are suitable for use include, but are not limited to, a monoclonal antibody to VEGF; a soluble VEGFR (see, e.g., Takayama et al. (2000) Cancer Res. 60:2169-2177; Mori et al. (2000) Gene Ther. 7:1027-1033; and Mahasreshti et al. (2001) Clin. Cancer Res. 7:2057-2066); a monoclonal antibody to VEGFR-2 (see, e.g., Prewett et al. (1999) Cancer Res. 59:5209-5218; Witte et al. (1998) Cancer Metastasis Rev. 17:155-161; Brekken et al. (2000) Cancer Res. 60:5117-5124; Kunkel et al. (2001) Cancer Res. 61:6624-6628); a soluble VEGFR as disclosed in U.S. Patent Publication No. 20030181377; an antibody to VEGFR as disclosed in U.S. Patent Publication No. 20030175271; a chimeric VEGF antagonist that includes an Ig domain from a VEGF receptor-1 (VEGFR1), an Ig domain from a VEGF receptor-2 (VEGFR2), and a dimerization domain or multimerization domain, as described in, e.g., Holash et al. ((2002) Proc. Natl. Acad. Sci. USA 99:11393-11398); and the like.

Whether angiogenesis is reduced can be determined using any method known in the art, including, e.g., reduction of neovascularization into implants impregnated with relaxin; reduction of blood vessel growth in the cornea or anterior eye chamber; reduction of endothelial cell proliferation, migration or tube formation in vitro; the chick chorioallantoic membrane assay; the hamster cheek pouch assay; the polyvinyl alcohol sponge disk assay; and the like. Such assays are well known in the art and have been described in numerous publications, including, e.g., Auerbach et al. ((1991) Pharmac. Ther. 51: 1-11), and references cited therein.

Fibrosis

In some embodiments, a subject method is effective to reduce fibrosis by at least about 5%, at least about 10%, at least about 20%, at least about 25%, at least about 50%, at least about 75%, at least about 85%, or at least about 90%, when compared to a suitable control. Thus, in these embodiments, “effective amounts” of an active agent are amounts of active agent that alone or in combination with other therapy for a fibrotic disorder are sufficient to reduce fibrosis by at least about 5%, at least about 10%, at least about 20%, at least about 25%, at least about 50%, at least about 75%, at least about 85%, or at least about 90%, when compared to a suitable control. In an experimental animal system, a suitable control may be a genetically identical animal not treated with the subject drug therapy. In non-experimental systems, a suitable control may be degree of fibrosis existing before treatment with a ppGalNAcT inhibitor. Other suitable controls may be a placebo control.

In some embodiments, a subject method for reducing fibrosis or treating a fibrotic disorder in an individual involves administering to an individual in need thereof an effective amount of a ppGalNAcT inhibitor, and an effective amount of a second therapeutic agent to treat a fibrotic disorder. Suitable second therapeutic agents include anti-inflammatory agents; interferon-gamma; corticosteroids; and the like.

Whether fibrosis is reduced can be determined by any known method, which will depend, in part, on the organ affected. As one non-limiting example, where the fibrosis is lung fibrosis, parameters of lung function include, but are not limited to, forced vital capacity (FVC); forced expiratory volume (FEV₁); total lung capacity; partial pressure of arterial oxygen at rest; partial pressure of arterial oxygen at maximal exertion. Lung function can be measured using any known method, including, but not limited to spirometry.

Cancer

In some embodiments, a subject method is effective to reduce the growth rate of a tumor by at least about 5%, at least about 10%, at least about 20%, at least about 25%, at least about 50%, at least about 75%, at least about 85%, or at least about 90%, up to total inhibition of growth of the tumor, when compared to a suitable control. Thus, in these embodiments, “effective amounts” of an active agent are amounts of active agent that alone or in combination with other therapy for cancer are sufficient to reduce tumor growth rate by at least about 5%, at least about 10%, at least about 20%, at least about 25%, at least about 50%, at least about 75%, at least about 85%, or at least about 90%, up to total inhibition of tumor growth, when compared to a suitable control. In an experimental animal system, a suitable control may be a genetically identical animal not treated with the subject drug therapy. In non-experimental systems, a suitable control may be the tumor growth rate existing before administering the subject ppGalNAcT inhibitor (alone or in combination with at least one additional anti-neoplastic agent). Other suitable controls may be a placebo control.

Whether growth of a tumor is inhibited can be determined using any known method, including, but not limited to, a proliferation assay; a ³H-thymidine uptake assay; and the like. Whether tumor mass is decreased can be determined using any known method, including, but not limited to, magnetic resonance imaging of the tumor, biopsy, and the like.

The methods are useful for treating a wide variety of cancers, including carcinomas, sarcomas, leukemias, and lymphomas.

Carcinomas that can be treated using a subject method include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelieal carcinoma, and nasopharyngeal carcinoma, etc.

Sarcomas that can be treated using a subject method include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated using a subject method include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL, prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; and the like.

Standard cancer therapies include surgery (e.g., surgical removal of cancerous tissue), radiation therapy, bone marrow transplantation, chemotherapeutic treatment, biological response modifier treatment, and certain combinations of the foregoing, as described above.

In one embodiment, the invention provides a method of treating cancer by co-administering a ppGalNAcTase inhibitor as an adjuvant to any therapy in which the cancer patient receives treatment with at least one additional antineoplastic drug, where the additional drug is an alkylating agent. In some embodiments, the alkylating agent is a nitrogen mustard. In other embodiments, the alkylating agent is an ethylenimine. In still other embodiments, the alkylating agent is an alkylsulfonate. In additional embodiments, the alkylating agent is a triazene. In further embodiments, the allkylating agent is a nitrosourea.

In another embodiment, the invention provides a method of treating cancer by co-administering a ppGalNAcTase inhibitor as an adjuvant to any therapy in which the cancer patient receives treatment with at least one additional antineoplastic drug, where the additional drug is an antimetabolite. In some embodiments, the antimetabolite is a folic acid analog, such as methotrexate. In other embodiments, the antimetabolite is a purine analog, such as mercaptopurine, thioguanine and axathioprine. In still other embodiments, the antimetabolite is a pyrimidine analog, such as 5FU, UFT, capecitabine, gemcitabine and cytarabine.

In another embodiment, the invention provides a method of treating cancer by co-administering a ppGalNAcTase inhibitor as an adjuvant to any therapy in which the cancer patient receives treatment with at least one additional antineoplastic drug, where the additional drug is a vinca alkyloid. In some embodiments, the vinca alkaloid is a taxane, such as paclitaxel. In other embodiments, the vinca alkaloid is a podophyllotoxin, such as etoposide, teniposide, ironotecan, and topotecan.

In another embodiment, the invention provides a method of treating cancer by co-administering a ppGalNAcTase inhibitor as an adjuvant to any therapy in which the cancer patient receives treatment with at least one additional antineoplastic drug, where the additional drug is an antineoplastic antibiotic. In some embodiments, the antineoplastic antibiotic is doxorubicin.

In another embodiment, the invention provides a method of treating cancer by co-administering a ppGalNAcTase inhibitor as an adjuvant to any therapy in which the cancer patient receives treatment with at least one additional antineoplastic drug, where the additional drug is a platinum complex. In some embodiments, the platinum complex is cisplatin. In other embodiments, the platinum complex is carboplatin.

In one embodiment, the invention provides a combination therapy comprising administering a ppGalNAcTase inhibitor as an adjuvant to any therapy in which the cancer patient receives treatment with least one additional antineoplastic drug, where the additional drug is a tyrosine kinase inhibitor. In some embodiments, the tyrosine kinase inhibitor is a receptor tyrosine kinase (RTK) inhibitor, such as type I receptor tyrosine kinase inhibitors (e.g., inhibitors of epidermal growth factor receptors), type II receptor tyrosine kinase inhibitors (e.g., inhibitors of insulin receptor), type III receptor tyrosine kinase inhibitors (e.g., inhibitors of platelet-derived growth factor receptor), and type IV receptor tyrosine kinase inhibitors (e.g., fibroblast growth factor receptor). In other embodiments, the tyrosine kinase inhibitor is a non-receptor tyrosine kinase inhibitor, such as inhibitors of src kinases or janus kinases.

In another embodiment, the invention provides a combination therapy comprising administering a ppGalNAcTase inhibitor as an adjuvant to any therapy in which the cancer patient receives treatment with least one additional antineoplastic drug, where the additional drug is an inhibitor of a receptor tyrosine kinase involved in growth factor signaling pathway(s). In some embodiments, the inhibitor is genistein. In other embodiments, the inhibitor is an EGFR tyrosine kinase-specific antagonist, such as IRESSA™ gefitinib (ZD18398; Novartis), TARCEVA™ erolotinib (OSI-774; Roche; Genentech; OSI Pharmaceuticals), or tyrphostin AG1478 (4-(3-chloroanilino)-6,7-dimethoxyquinazoline. In still other embodiments, the inhibitor is any indolinone antagonist of Flk-1/KDR (VEGF-R2) tyrosine kinase activity described in U.S. Patent Application Publication No. 2002/0183364 A1, such as the indolinone antagonists of Flk-1/KDR (VEGF-R2) tyrosine kinase activity disclosed in Table 1 on pages 4-5 thereof. In further embodiments, the inhibitor is any of the substituted 3-[(4,5,6,7-tetrahydro-1H-indol-2-yl) methylene]-1,3-dihydroindol-2-one antagonists of Flk-1/KDR (VEGF-R2), FGF-R1 or PDGF-R tyrosine kinase activity disclosed in Sun, L., et al., J. Med. Chem., 43(14): 2655-2663 (2000). In additional embodiments, the inhibitor is any substituted 3-[(3- or 4-carboxyethylpyrrol-2-yl) methylidenyl]indolin-2-one antagonist of Flt-1 (VEGF-R1), Flk-1/KDR (VEGF-R2), FGF-R1 or PDGF-R tyrosine kinase activity disclosed in Sun, L., et al., J. Med. Chem., 42(25): 5120-5130 (1999).

In another embodiment, the invention provides a combination therapy comprising administering a ppGalNAcTase inhibitor as an adjuvant to any therapy in which the cancer patient receives treatment with least one additional antineoplastic drug, where the additional drug is an inhibitor of a non-receptor tyrosine kinase involved in growth factor signaling pathway(s). In some embodiments, the inhibitor is an antagonist of JAK2 tyrosine kinase activity, such as tyrphostin AG490 (2-cyano-3-(3,4-dihydroxyphenyl)-N-(benzyl)-2-propenamide). In other embodiments, the inhibitor is an antagonist of bcr-abl tyrosine kinase activity, such as GLEEVEC™ imatinib mesylate (STI-571; Novartis).

In another embodiment, the invention provides a combination therapy comprising administering a ppGalNAcTase inhibitor as an adjuvant to any therapy in which the cancer patient receives treatment with least one additional antineoplastic drug, where the additional drug is a serine/threonine kinase inhibitor. In some embodiments, the serine/threonine kinase inhibitor is a receptor serine/threonine kinase inhibitor, such as antagonists of TGF-β receptor serine/threonine kinase activity. In other embodiments, the serine/threonine kinase inhibitor is a non-receptor serine/threonine kinase inhibitor, such as antagonists of the serine/threonine kinase activity of the MAP kinases, protein kinase C (PKC), protein kinase A (PKA), or the cyclin-dependent kinases (CDKs).

In another embodiment, the invention provides a combination therapy comprising administering a ppGalNAcTase inhibitor as an adjuvant to any therapy in which the cancer patient receives treatment with least one additional antineoplastic drug, where the additional drug is an inhibitor of one or more kinases involved in cell cycle regulation. In some embodiments, the inhibitor is an antagonist of CDK2 activation, such as tryphostin AG490 (2-cyano-3-(3,4-dihydroxyphenyl)-N-(benzyl)-2-propenamide). In other embodiments, the inhibitor is an antagonist of CDK1/cyclin B activity, such as alsterpaullone. In still other embodiments, the inhibitor is an antagonist of CDK2 kinase activity, such as indirubin-3′-monoxime. In additional embodiments, the inhibitor is an ATP pool antagonist, such as lometrexol (described in U.S. Patent Application Publication No. 2002/0156023 A1).

In another embodiment, the invention provides a combination therapy comprising administering a ppGalNAcTase inhibitor as an adjuvant to any therapy in which the cancer patient receives treatment with least one additional antineoplastic drug, where the additional drug is an a tumor-associated antigen antagonist, such as an antibody antagonist. In some embodiments involving the treatment of HER2-expressing tumors, the tumor-associated antigen antagonist is an anti-HER2 monoclonal antibody, such as HERCEPTIN™ trastuzumab. In some embodiments involving the treatment of CD20-expressing tumors, such as B-cell lymphomas, the tumor-associated antigen antagonist is an anti-CD20 monoclonal antibody, such as RITUXAN™ rituximab.

In another embodiment, the invention provides a combination therapy comprising administering a ppGalNAcTase inhibitor as an adjuvant to any therapy in which the cancer patient receives treatment with least one additional antineoplastic drug, where the additional drug is a tumor growth factor antagonist. In some embodiments, the tumor growth factor antagonist is an antagonist of epidermal growth factor (EGF), such as an anti-EGF monoclonal antibody. In other embodiments, the tumor growth factor antagonist is an antagonist of epidermal growth factor receptor erbB1 (EGFR), such as an anti-EGFR monoclonal antibody inhibitor of EGFR activation or signal transduction, including ERBITUX™ cetuximab, or a small molecule antagonist of EGFR activation or signal transduction, such as IRESSA™ gefitinib and TARCEVA™ erolotinib.

In another embodiment, the invention provides a combination therapy comprising administering a ppGalNAcTase inhibitor as an adjuvant to any therapy in which the cancer patient receives treatment with least one additional antineoplastic drug, where the additional drug is an Apo-2 ligand agonist. In some embodiments, the Apo-2 ligand agonist is any of the Apo-2 ligand polypeptides described in WO 97/25428.

In another embodiment, the invention provides a combination therapy comprising administering a ppGalNAcTase inhibitor as an adjuvant to any therapy in which the cancer patient receives treatment with least one additional antineoplastic drug, where the additional drug is an anti-angiogenic agent. In some embodiments, the anti-angiogenic agent is a vascular endothelial cell growth factor (VEGF) antagonist, such as an anti-VEGF monoclonal antibody, e.g. AVASTIN™ bevacizumab (Genentech). In other embodiments, the anti-angiogenic agent is an antagonist of VEGF-R1, such as an anti-VEGF-R1 monoclonal antibody. In other embodiments, the anti-angiogenic agent is an antagonist of VEGF-R2, such as an anti-VEGF-R2 monoclonal antibody. In other embodiments, the anti-angiogenic agent is an antagonist of basic fibroblast growth factor (bFGF), such as an anti-bFGF monoclonal antibody. In other embodiments, the anti-angiogenic agent is an antagonist of bFGF receptor, such as an anti-bFGF receptor monoclonal antibody. In other embodiments, the anti-angiogenic agent is an antagonist of TGF-β, such as an anti-TGF-β monoclonal antibody. In other embodiments, the anti-angiogenic agent is an antagonist of TGF-β receptor, such as an anti-TGF-β receptor monoclonal antibody. In other embodiments, the anti-angiogenic agent is a retinoic acid receptor (RXR) ligand, such as any RXR ligand described in U.S. Patent Application Publication No. 2001/0036955 A1 or in any of U.S. Pat. No. 5,824,685; 5,780,676; 5,399,586; 5,466,861; 4,810,804; 5,770,378; 5,770,383; or 5,770,382. In still other embodiments, the anti-angiogenic agent is a peroxisome proliferator-activated receptor (PPAR) gamma ligand, such as any PPAR gamma ligand described in U.S. Patent Application Publication No. 2001/0036955 A1.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s, second(s); min, minute(s); hr, hour(s); aa, amino acid(s); kb, kilobase(s); bp, base pair(s); nt, nucleotide(s); and the like.

### Example 1

### Identification and Characterization of Small Molecule Inhibitors of Mucin-Type O-Linked Glycosylation

Experimental Procedures

Methyl 2-acetamido-2-deoxy-β-D-glucopyranoside (βOMeGlcNAc), pyruvate kinase, lactate dehydrogenase, phosphoenolpyruvate, NADH and uridine diphosphate N-acetyl-α-galactosamine (UDP-GalNAc) were purchased from Sigma. Helix pomatia agglutinin (HPA) conjugated to horse radish peroxidase (HRP) (HPA-HRP), fluorescein isothiocyanate-labeled HPA (FITC-HPA), FITC-labeled concanavalin-A (FITC-Con A) and FITC-jacalin were from E-Y Laboratories. Transparent 96-well Reacti-Bind NeutrAvidin-coated plates and the HRP substrate 3,3′,5,5′-tetramethyl benzidine (TMB) were purchased from Pierce. Doxorubicin hydrochloride, campothecin, UDP-Gal, N-acetylactosamine (LacNAc), bovine β1-4GalT and porcine α1-3GalT were purchased from Calbiochem. Aldehydes were purchased from Aldrich, ChemDiv or ChemBlock as listed in the Supplementary Material.

Enzyme assays were quantified using a Molecular Devices UV/Vis 96-well plate reader (SpectraMax 190). Reverse phase-high performance liquid chromatography (RP-HPLC) was performed using a Rainin Dynamax SD-200 HPLC system with 230 nm detection on a Microsorb C-18 analytical column (4.6×250 mm), at a flow rate of 1 mL/min or a preparative column (25×250 mm) at a flow rate of 20 mL/min.

All ¹H and ¹³C NMR spectra were recorded on a Bruker DRX 500 MHz NMR spectrometer. Chemical shifts are reported in ppm relative to tetramethylsilane. Coupling constants (J) are reported in Hz. Fast atom bombardment (FAB) and electrospray (ES) mass spectra were obtained at the UC Berkeley Mass Spectrometry Laboratory.

Jurkat cells were grown in RPMI-1640 media supplemented with 10% FCS, 100 units/mL penicillin and 0.1 mg/mL streptomycin. HEK 293T cells were grown in MEM supplemented with 10% FCS, 2 mM L-glutamine and Earle's BSS adjusted to contain 1.5 g/L sodium bicarbonate, 0.1 mM non-essential amino acids, and 1.0 mM sodium pyruvate. Cells were incubated in a 5% CO₂ humidified incubator at 37° C.

Solid-Phase Peptide Synthesis

The biotinylated peptides and glycopeptides were generated by standard Fmoc-based solid-phase peptide synthesis methods using N^{α}-Fmoc-Thr(Ac₃-α-D-GalNAc)—OH (Winans et al. (1999) Biochemistry 38:11700-11710) as a glycosylated amino acid building block. Peptides were purified by RP-HPLC eluting with 10-40% MeCN/H₂O with 0.1% TFA. C-terminal biotinylated EA2 (4): LRMS (ES): calcd. for C₇₁H₁₁₉N₁₉O₂₅S (M+H⁺) 1670.9, found 1670.7. C-terminal biotinylated EA2* (5): LRMS (ES): calcd. for C₇₉H₁₃₂N₂₀O₃₀S (M+H⁺) 1874.1, found 1874.1. MUC5AC** LRMS (ES): calcd. for C₇₉H₁₃₂N₁₉O₃₅ (M+H⁺) 1907.0, found 954.5 (M+2H⁺).

Expression of ppGalNAcTs

COS-7 cells were transiently transfected with plasmids encoding truncated secreted ppGalNAcTs-1 to −5, -7, -10 and -11 as previously described. Hagen et al. (1997) J. Biol. Chem. 272:13843-13848. The crude conditioned medium was used as the enzyme source.

ELLA for ppGalNAcTs

Standard reaction conditions for ppGalNAcT assays were as follows. The reaction mixture contained the following components in a final volume of 25 μL: 10 mM MnCl₂, 40 mM sodium cacodylate, 40 mM β-mercaptoethanol, 0.1% Triton X-100, pH 6.5, 5 μL of conditioned media, and various concentrations of EA2 peptide 4, UDP-GalNAc and inhibitor. All uridine-based compounds were dissolved in dimethyl sulfoxide (DMSO). The concentration of DMSO in the reaction mixture was limited to 2% (v/v), as higher concentrations decreased ppGalNAcT activity. For inhibitor assays, reaction mixtures for positive controls also contained 2% DMSO. Unless otherwise stated, inhibitor assays were performed using 20 μM UDP-GalNAc and 50 μM EA2 peptide 4, the K_{M} values for both substrates. Reactions were incubated at 37° C. and were terminated by the addition of 10 μL of 0.1 M EDTA. All experiments were performed in duplicate. Reaction rates were linear over the period monitored (20-30 min).

96-Well NeutrAvidin-coated plates were prewashed 3 times with 200 μL of wash buffer (25 mM Tris, 150 mM NaCl, 0.1% bovine serum albumin (BSA) (w/v), and 0.05% Tween (v/v), pH 7.2). Reactions were then transferred to the 96-well plates, incubated for 1 hour at room temperature (rt) and subsequently washed 3 times with 200 μL of phosphate-buffered saline (PBS) containing 0.5% BSA and 0.05% Tween, pH 7.1. A 100-μL solution of HPA-HRP in standard PBS buffer (1 μg/mL, pH 7.4) was added and the reaction was incubated for 1 h at rt. After 3 washes with 200 μL of PBS, bound HPA-HRP was quantified by the addition of 100 μL of TMB peroxide solution. The solutions were incubated for 5-15 min in the dark at rt. HRP activity was terminated by the addition of 50 μL of 2 N H₂SO₄ and the resulting solution was analyzed at 450 nm using a UV/Vis microtiter plate reader. The amount of product generated by the ppGalNAcT was extrapolated from absorbance values using an equation fit to the standard curve derived from Origin 6.1 software.

Radiolabel Capture Assay for rppGalNAcT-5, -7 and -10

Radiolabel capture assays using ¹⁴C-labeled UDP-GalNAc were performed as previously described with various concentrations of 1-68A or 2-68A in 2% DMSO. Ten Hagen et al. (2001) J. Biol. Chem. 276:17395-17404.

Continuous Assay for β1-4GalT and α1-3GalT Activity

The enzymatic reactions contained the following components in a final volume of 100 μL: 20 mM MnCl₂, 100 mM sodium cacodylate, pH 6.5, 5 U of pyruvate kinase, 5 U of lactate dehydrogenase, 2 mM PEP, 0.2 mM NADH with 0.4 mU of β1-4GalT, 1 mM βOMeGlcNAc, 25 μM UDP-Gal or 1.0 mU of α1-3GalT, 1 mM LacNAc, 100 μM UDP-Gal and various concentrations of 1-68A or 2-68A in 2.5% DMSO. The reaction mixtures were incubated at 37° C. for 10 min before the reaction was initiated by the addition of UDP-Gal. The change in absorbance was monitored over 20 min at 340 nm.

Resynthesis of 1-68A and 2-68A

The aldehyde (0.06 mmol) and the corresponding uridine analog (0.08 mmol) were stirred for 16 h at rt in 1% AcOH/DMSO (0.6 mL) in the dark. RP-HPLC purification eluting with a gradient of 15-80% MeCN/H₂O afforded compound 1-68A (16 mg, 0.04 mmol) in 67% yield as an off-white solid and compound 2-68A (18 mg, 0.04 mmol) in 67% yield as an off-white solid.

1-68A ¹H NMR (500 MHz, CD₃OD): δ 8.26 (s, 1), 7.74 (d, 1, J=8.1), 6.73 (d, 1, J=8.5), 6.42 (d, 1, J=8.5), 5.88 (d, 1, J=4.2), 5.64 (d, 1, J=8.1), 4.47 (app d, 1, J=12.5), 4.37 (app d, 1, J=12.3), 4.21-4.16 (m, 3H). ¹³C NMR (125 MHz, MeOD): δ 164.6, 151.6, 150.8, 148.3, 146.0, 140.8, 132.5, 121.0, 109.2, 107.3, 101.3, 89.8, 82.8, 73.8, 73.2, 69.8. HRMS (FAB): calcd. for C₁₆H₁₇N₃O₉ (M+H⁺) 396.1039, found 396.1043.

2-68A ¹H NMR (500 MHz, CD₃OD): δ 8.33 (s, 1), 7.60 (d, 1, J=8.1), 6.73 (d, 1, J=8.6), 6.41 (d, 1, J=8.5), 5.70 (d, 1, J=4.8), 5.60 (d, 1, J=8.0), 4.63 (app s, 1), 4.21 (app t, 1, J=5.0), 4.04-4.02 (m, 2), 3.65-3.54 (m, 2). ¹³C NMR (125 MHz, MeOD): δ 171.1, 164.6, 153.0, 150.8, 148.6, 146.0, 141.8, 132.5, 121.2, 108.9, 107.5, 101.4, 91.0, 82.2, 73.2, 72.4, 70.8, 40.4. HRMS (FAB): calcd. for C₁₈H₂₀N₄O₁₀ (M+H⁺) 453.1253, found 453.1258.

Evaluation of 1-68A, 2-68A, Doxorubicin and Campothecin in Jurkat Cells

Jurkat cells were seeded at 250,000 cells/well (determined by Coulter cell counter) in 12-well polystyrene tissue culture plates in 1.0 mL of media and treated with various concentrations of 1-68A or 2-68A from 50 mM dimethyl sulfoxide (DMSO) stock solutions or doxorubicin or campothecin from 1 mM DMSO stock solutions. After 2 d, cells were harvested, washed twice with PBS buffer (PBS, pH 7.4, 0.1% FCS, 0.1% NaN₃ w/v) and stained with FITC-HPA (1 μg/mL) or FITC-Con A (5 μg/mL with 1 mM CaCl₂) in PBS, pH 7.4 in the dark for 1 h at 4° C. Cells were washed twice with PBS buffer, resuspended in 300 μL of PBS buffer and analyzed by flow cytometry (FacsCaliber, BD Instruments). Annexin-V staining was performed according to the manufacturer's protocols (Invitrogen).

Evaluation of 1-68A and 2-68A in HEK 293T Cells

HEK 293T cells were seeded at 20,000 cells/mL in a 24-well plate (Costar) fit with glass cover slips (Fisher). After 24 h of incubation with 100 μM 1-68A, cells were washed twice with PBS (pH 7.4) and fixed with 4% paraformaldehyde-PBS for 1 h at rt. The cells were washed twice with PBS and incubated with permeabilization solution (0.1% Triton X-100 in 0.1% sodium citrate, freshly prepared) for 2 min on ice. After the cells were washed twice with PBS, 50 μL of TUNEL reaction mixture (1:10 dilution, Boehringer Mannheim) was added. The plate was covered with parafilm to avoid evaporative loss and incubated in a humidified atmosphere for 1 h at 37° C. in the dark. The cells were then rinsed three times with PBS and blocked with 2% BSA in PBST (PBS, 0.05% Tween 20) for 30 min at rt. Following one wash with PBS, the cells were incubated with 100 μL of FITC-jacalin (4 μg/mL in PBST) for 1 h at 37° C. in the dark. The cells were washed three times with PBS, counterstained with Hoechst 33342 (Molecular Probes) and mounted with Fluoromount-G aqueous medium (Electron Microscopy Sciences). Slides were analyzed by fluorescence microscopy (Zeiss, using Openlab software).

Results

Development and Validation of a High-Throughput Assay for ppGalNAcTs

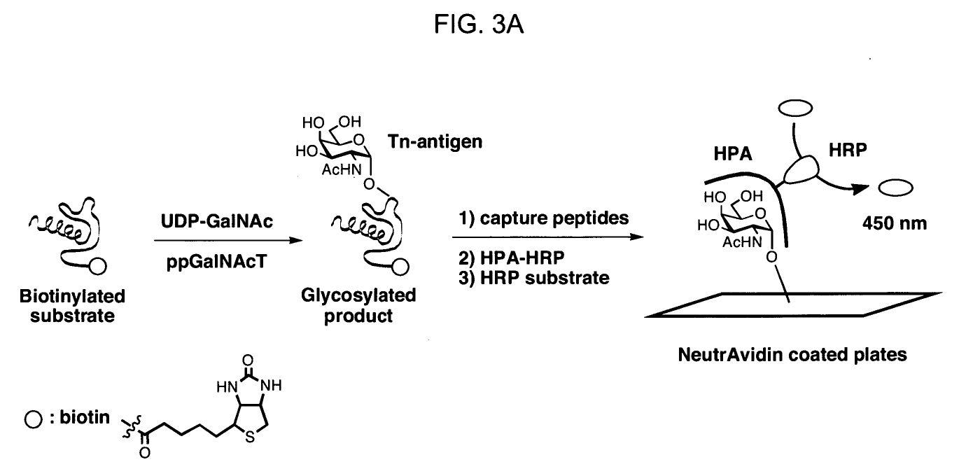

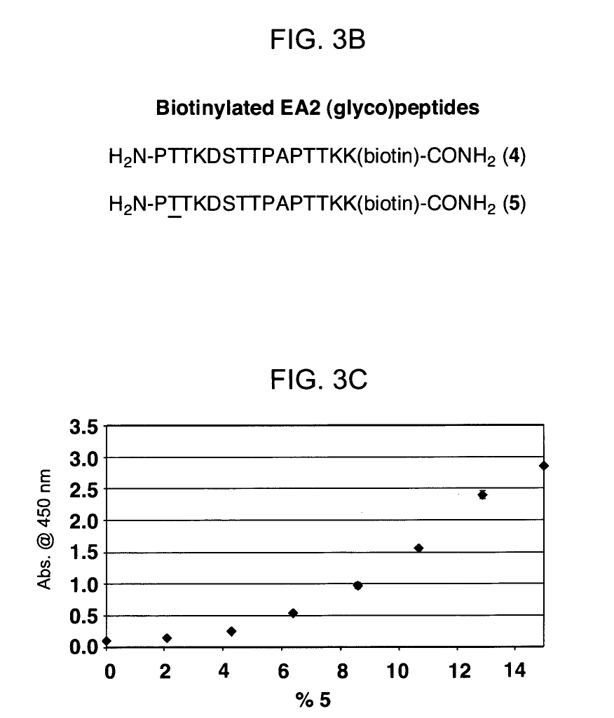

In order to screen the library in high-throughput format, a non-radioactive enzyme linked lectin assay (ELLA) for ppGalNAcTs was developed. A schematic diagram of the assay is shown in FIG. 3A. As an acceptor substrate, the EA2 peptide (PTTDSTTPAPTTK; SEQ ID NO:12), a fragment of rat submandibular mucin, was chosen. Albone et al. (1994) J. Biol. Chem. 269:16845-16852. This peptide has been previously shown to be an efficient substrate for murine (m) ppGalNAcT-1 and is preferentially glycosylated at the fourth Thr residue from the N-terminus (underlined). Ten Hagen et al. (2001, supra). Biotinylation of EA2 allowed capture of (glyco)peptides onto 96-well NeutrAvidin-coated plates. The GalNAc-modified glycopeptide product could be detected using the α-GalNAc-specific lectin Helix pomatia agglutinin (HPA) (Hammarstrom et al. (1977) Biochemistry 16:2750-2755) conjugated to horseradish peroxidase (HRP). The bound HRP was quantified by addition of a chromogenic substrate.

In order to correlate the change in absorbance produced by HRP activity to the enzymatic activity of mppGalNAcT-1, a standard curve for the ELLA response was generated. Peptide 4 (PTTKDSTTPAPTTKK; SEQ ID NO:16) and glycopeptide 5 (PTTKDSTTPAPTTKK; SEQ ID NO:17, where the underlined T is glycosylated), biotinylated at the C-terminus, were constructed by Fmoc-based solid-phase peptide synthesis using previously described methods (FIG. 3B). Winans et al. (1999) Biochemistry 38:11700-11710. Peptide 4 and glycopeptide 5 were combined at various percentages, captured on NeutrAvidin-coated 96-well microtiter plates and detected using the HPA-HRP conjugate. The standard curve derived from this experiment correlates the percentage of NeutrAvidin sites occupied by the glycopeptide with the observed signal. The absolute quantity of immobilized glycopeptide can be determined based on the known binding capacity of the NeutrAvidin-coated plates (60 pmol/well). The standard curve showed a dose-dependent increase in signal over a range of 0-15% 5 (FIG. 3C). The signal reached a plateau at higher concentrations of 5, which was attributed to saturation of lectin binding. The signal to noise ratio observed at 15% 5 (9 pmol) was 30-fold above background. Thus, the assay can readily detect low picomole amounts of the product of the ppGalNAcT reaction.

To validate the ELLA, the kinetic parameters of mppGalNAcT-1 was measured with UDP-GalNAc, EA2 peptide 4 and UDP (details are provided in the Supplementary Material). The K_{M} values of UDP-GalNAc and EA2 peptide 4 were determined to be 13.9±1.8 μM and 48.0±4.0 μM, respectively. The K_{I} value for the product UDP was 251.1±78.0 μM. These values are similar to those previously determined using a radiolabel capture assay. Ten Hagen et al. (1998) J. Biol. Chem. 273:27749-27754; and Wragg et al. (1995) J. Biol. Chem. 270:16974-16954.

Preliminary Screening of the Uridine-Based Library with mppGalNAcT-1

Using the ELLA, preliminary screens of the 1338-member uridine-based library were performed (Winans et al. (2002) Chem. Biol. 9:113-129) at 40 μM with mppGalNAcT-1. From the preliminary screens, 32 initial hits that displayed over 70% inhibition were identified. After rescreening these initial hits at 8 μM and resynthesis of confirmed hits, two mppGalNAcT-1 inhibitors 1-68A and 2-68A were identified (FIG. 4A). The compounds comprised the same aldehyde component (68A) linked via an oxime to two different uridine scaffolds, 1 and 2. Interestingly, the aldehyde component has a trihydroxybenzene functionality that resembles a monosaccharide.

Kinetic Analysis of Uridine-Based Inhibitors with mppGalNAcT-1

To determine the mode of inhibition of 1-68A and 2-68A, their inhibitory activity versus both substrates UDP-GalNAc and EA2 peptide 4 was evaluated. The K_{I} values for 1-68A and 2-68A were determined to be 7.8±0.1 μM and 7.8±1.0 μM versus UDP-GalNAc, respectively (FIG. 4B), with both sharing competitive behavior with respect to UDP-GalNAc. Their binding affinities were approximately 2-fold greater than UDP-GalNAc (K_{M}=14 μM) and 30-fold greater than UDP (K_{I}=250 μM). Both compounds appeared to be non-competitive with respect to EA2 peptide 4, a finding consistent with a random sequential mechanism reported by Wragg et al. (supra).

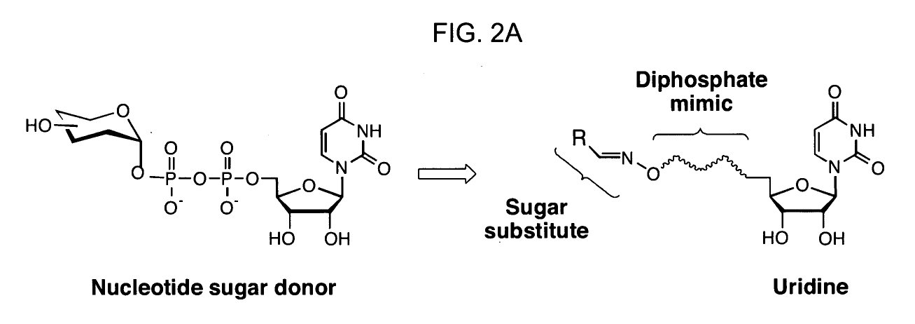

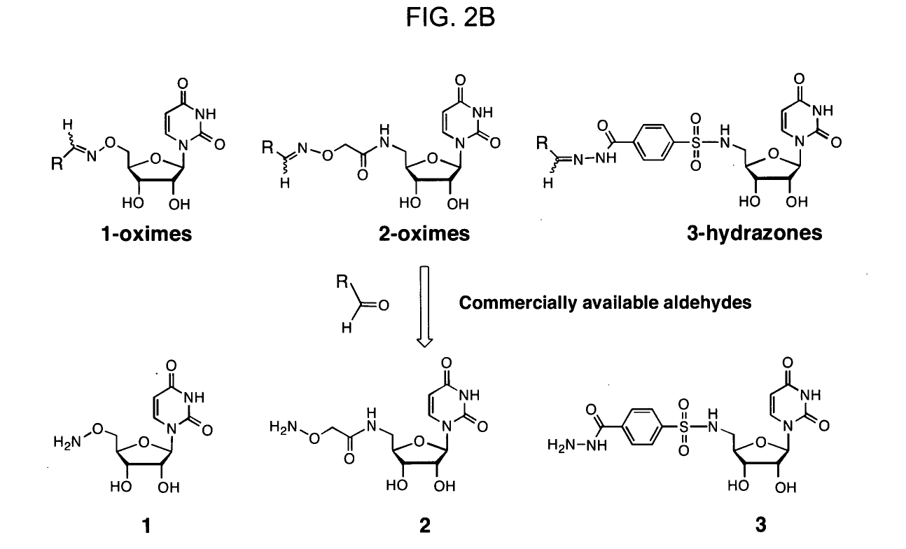

To determine the contributions of the uridine and aldehyde components to binding, aldehyde 68A and the parent aminooxy uridine analogs 1 and 2 were assayed for inhibitory activity. While uridine analogs 1 and 2 showed no inhibition at concentrations up to 400 μM, compound 68A exhibited competitive inhibitory activity with a K_{I} value of 34.3±5.5 μM (FIG. 4B). These data suggest that the binding affinity of 68A was increased approximately 5-fold when coupled to uridine analogs 1 or 2. It is interesting to note that the K_{I} values for compounds 1-68A and 2-68A are similar despite the different linker lengths, suggesting that the aldehyde component contributes significantly to binding. However, the adduct of aldehyde 68A with uridine analog 3 (FIG. 2B), the longest of the three uridine linkers, showed no inhibitory activity in the secondary screen at 8 μM, suggesting the structure and/or length of the linker is a critical determinant of binding.

Inhibitory Activity Against Other Related Enzymes

To evaluate the activities of compounds 1-68A and 2-68A with other ppGalNAcT isoforms, IC₅₀ measurements for both compounds were performed with ppGalNAcTs 1-5, -7, -10 and -11 (Entries 1-8, Table 1). Their inhibitory activities were similar with all ppGalNAcT isoforms tested. Thus, 1-68A and 2-68A appear to be general inhibitors of the ppGalNAcT family.

TABLE 1

| | 2.1-68A (μM) | 2.2-68A (μM) |
|---|---|---|
| 1) mppGalNAcT-1 | 21 ± 1 | 24 ± 2 |
| 2) mppGalNAcT-2 | 15 ± 1 | 18 ± 2 |
| 3) mppGalNAcT-3 | 40 ± 2 | 38 ± 4 |
| 4) mppGalNAcT-4 | 30 ± 5 | 20 ± 5 |
| 5) rppGalNAcT-5 | 20 ± 2 | 26 ± 9 |
| 6) rppGalNAcT-7 | 22 ± 2 | 27 ± 2 |
| 7) rppGalNAcT-10 | 7 ± 1 | 6 ± 1 |
| 8) mppGalNAcT-11 | 39 ± 3 | 32 ± 3 |
| 9) β1-4GalT | >500 | >500 |
| 10) α1-3GalT | >500 | >500 |

Table 1. IC₅₀ values for 1-68A and 2-68A with ppGalNAcTs-1 to −5, -7, -10, -11, β1-4GalT and α1-3GalT. The requirement of glycopeptides as substrates for ppGalNAcT-7 and -10 precluded the use of the ELLA, as both the substrate and product bind HPA-HRP. Thus, the radiolabel capture assay was employed to measure the IC₅₀ values of 1-68A and 2-68A with ppGalNAcT-7 and -10 using MUC5AC (GTTPSPVPTTSTTSAP; SEQ ID NO:15) glycopeptide, previously shown to be substrate for both enzymes. Ten Hagen et al. (1999) J. Biol. Chem. 274:27867-27874; and Ten Hagen et al. (2001) J. Biol. Chem. 276:17395-17404. IC₅₀ values of 1-68A and 2-68A with ppGalNAcT-5 were performed with α-FLAG purified enzyme and radiolabel capture assay, due to low activity and stability of the crude enzyme. m=murine, r=rattus.

To determine the selectivity of the compounds among the broader family of UDP-sugar utilizing enzymes, 1-68A and 2-68A were tested against bovine β1-4galactosyltransferase (β1-4GalT) and porcine α1-3galactosyltransferase (α1-3GalT), using a previously reported continuous colorimetric assay. Fitzgerald et al. (1970) Anal. Biochem. 36:43-61. Neither compound was active against β1-4GalT or α1-3GalT at the highest concentration tested (500 μM) (Entries 9 and 10, Table 1). While every UDP-sugar utilizing enzyme in the vertebrate genome has not been evaluated, the lack of inhibitory activity of 1-68A and 2-68A against β1-4GalT and a 1-3 GalT demonstrates that these compounds are not general inhibitors of inverting or retaining glycosyltransferases. It should also be noted that 1-68A and 2-68A were not identified in screens of the uridine-based library against the UDP-GlcNAc/GalNAc C₄-epimerase (Winans and Bertozzi (2002) supra) or UDP-galactopyranose mutase (Scherman et al. (2003) Antimicrob. Agents Chemother. 47:378-382). Collectively, these observations suggest that 1-68A and 2-68A are selective inhibitors of the ppGalNAcT family and do not function as non-specific inhibitors of UDP-sugar utilizing enzymes.

Evaluation of 1-68A and 2-68A Inhibitory Activity in Cells

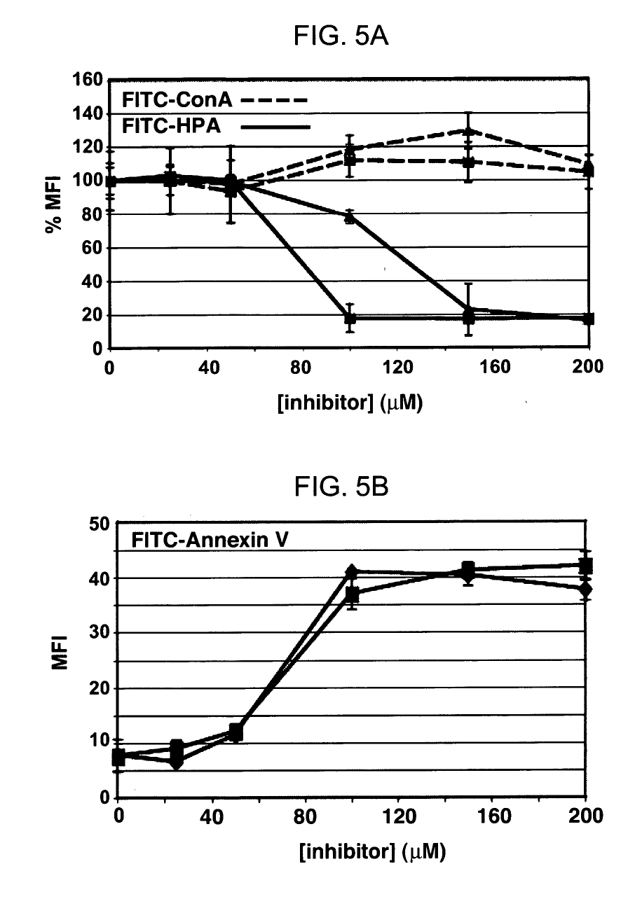

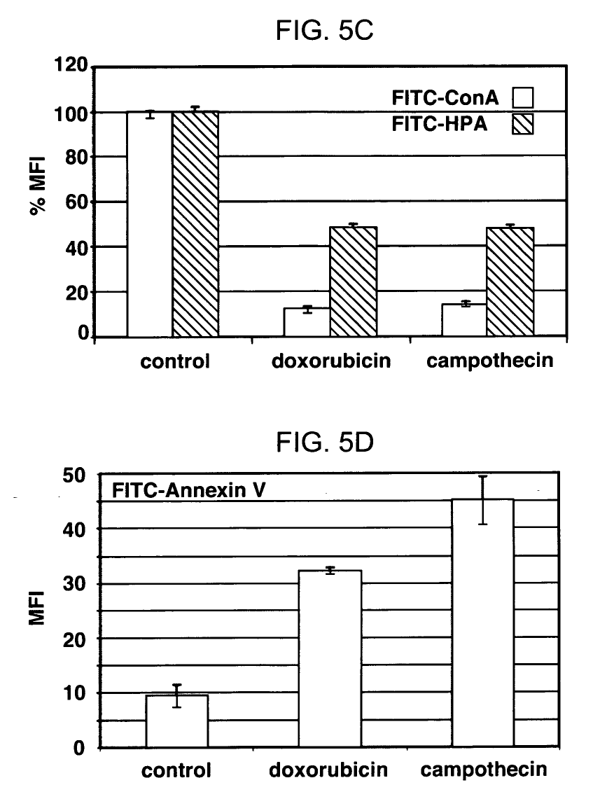

Having demonstrated that 1-68A and 2-68A inhibit the ppGalNAcTs in vitro, their effects on O-linked glycosylation in cells were evaluated. To directly monitor ppGalNAcT activities in cells, Jurkat cells (human T-cell lymphoma), which are known to produce only the Tn-antigen (FIG. 1) as their O-linked glycans, were chosen. Piller et al. (1990) J. Biol. Chem. 265:9264-9271. Changes in Tn-antigen expression on the surface of Jurkat cells were monitored by HPA binding followed by flow cytometry analysis. Con A staining of N-linked glycans was used as a control for non-specific inhibition of protein glycosylation. Baenziger and Fiete (1979) J. Biol. Chem. 254:2400-2407. As shown in FIG. 5A, both 1-68A and 2-68A inhibited HPA staining of Jurkat cells in a dose-dependent manner (EC₅₀˜80 μM) with no significant effect on Con A staining. However, forward and side scatter analysis of the cells treated with either compound for 2 days indicated that a morphological change characteristic of apoptosis had occurred. Indeed, Annexin-V staining of Jurkat cells treated with 1-68A or 2-68A confirmed the induction of apoptosis (FIG. 5B) at inhibitor concentrations that also abrogate HPA staining.

It is possible that compounds 1-68A and 2-68A induce apoptosis independently of their effects on O-linked glycosylation; and that changes in membrane architecture associated with the process affect lectin staining of cells. The effects of compounds known to induce apoptosis by glycosylation-independent mechanisms on lectin staining were analyzed. Jurkat cells were treated with the pro-apoptotic drugs doxorubicin and campothecin, which inhibit topoisomerases I and II, respectively, and evaluated for HPA, Con A and Annexin V staining. As shown in FIGS. 5C and 5D, doxorubicin and campothecin induced Annexin V binding at levels comparable to 1-68A. In contrast to 1-68A, doxorubicin and campothecin reduced both HPA and Con A staining of Jurkat cells. Thus, the physiological changes associated apoptosis alone cannot account for the selective reduction in HPA staining observed with 1-68A. Moreover, it is unlikely that the effects of 1-68A simply reflect a global disruption in metabolism, as one would expect a similar effect on N-linked glycan expression.

To determine if the inhibition of O-linked glycosylation and induction of apoptosis by 1-68A and 2-68A were specific to Jurkat cells, the effects of 1-68A on human embryonic kidney (HEK) 293T cells were evaluated. In this case, O-linked glycans on the cell surface were monitored by staining with jacalin, a lectin that binds core 1 structures (Galβ1,3-GalNAcα1-Ser/Thr). To evaluate apoptosis in HEK cells, TUNEL staining for DNA fragmentation was performed. Gavreli et al. (1992) J. Cell. Biol. 119:493-501. The results showed that 1-68A inhibits jacalin staining at 100 μM and increases TUNEL staining compared to untreated HEK cells. Thus, 1-68A appears to block O-linked glycosylation and induce apoptosis in HEK cells as well as in Jurkat cells.

The above-described experiments demonstrate that selective inhibitors of ppGalNAcT were identified, and that the inhibitors induce apoptosis in eukaryotic cells; Furthermore, a non-radioactive assay for inhibitors of ppGalNAcT was developed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An isolated compound that selectively inhibits enzymatic activity of a polypeptide N-acetyl-α-galactosaminyltransferase (ppGalNAcT).

2. The compound of claim 1, wherein the compound is of the generic formula #1.

3. The compound of claim 1, wherein the compound is of the generic formula #2.

4. The compound of claim 1, wherein the compound is of the generic formula #3.

5. The compound of claim 1, wherein the compound is designated 1-68A and has the structure shown in FIG. 4A.

6. The compound of claim 1, wherein the compound is designated 2-68A and has the structure shown in FIG. 4A.

7. The compound of claim 1, wherein the compound is designated 68A and shown in FIG. 4A.

8. A composition comprising a compound of claim 1.

9. A formulation comprising a compound of claim 1; and a pharmaceutically acceptable excipient.

10. A non-radioactive in vitro method of identifying agents that inhibit the enzymatic activity of a polypeptide N-acetyl-α-galactosaminyltransferase (ppGalNAcT) polypeptide, the method comprising:
a) contacting a ppGalNAcT polypeptide with a peptide substrate and a test agent;
b) determining the effect, if any, of the test agent on N-acetyl galactosamine (GalNAc) modification of the peptide substrate by the ppGalNAcT polypeptide, wherein said determining comprises detecting binding of a detectably labeled moiety that binds GalNAc-modified peptide substrate, wherein the detectable label is a non-radioactive label.

11. The method of claim 10, wherein the detectably labeled moiety is Helix pomatia agglutinin.

12. The method of claim 10, wherein the detectable label is an enzyme.

13. The method of claim 10, wherein the peptide substrate is immobilized on a solid support.

14. The method of claim 10, wherein the peptide substrate comprises the amino acid sequence PTTDSTTPAPTTK (SEQ ID NO:12).

15. A method for reducing mucin-type O-linked glycosylation in a eukaryotic cell, the method comprising contacting a cell with a compound of claim 1.

16. The method of claim 15, wherein the compound reduces cell proliferation.

17. The method of claim 15, wherein the cell is in in vitro cell culture.

18. The method of claim 15, wherein the cell is in a tissue in vitro.

19. A method of reducing tumor growth in an individual, the method comprising administering to an individual in need thereof a compound of claim 1.

20. The method of claim 19, further comprising administering at least one additional anti-neoplastic agent.


SEQUENCE LISTING (17 sequences)

SEQ ID NO: 1 - protein, 15 aa, Artificial Sequence
  ppGalNAcT substrate
GTTPAPVTTSTTSAP

SEQ ID NO: 2 - protein, 11 aa, Artificial Sequence
  ppGalNAcT substrate
PPDAATAAPLR

SEQ ID NO: 3 - protein, 16 aa, Artificial Sequence
  ppGalNAcT substrate
QTSSPNTGKTSTISTT

SEQ ID NO: 4 - protein, 24 aa, Artificial Sequence
  ppGalNAcT substrate
CPPTPSATTPAPPSSSAPPETTAA

SEQ ID NO: 5 - protein, 21 aa, Artificial Sequence
  ppGalNAcT substrate
QATEYEYLDYDFLPETEPPEM

SEQ ID NO: 6 - protein, 22 aa, Artificial Sequence
  ppGalNAcT substrate
CIRIQRGPGRAFVTIGKIGNMR

SEQ ID NO: 7 - protein, 11 aa, Artificial Sequence
  ppGalNAcT substrate
AHGVTSAPDTR

SEQ ID NO: 8 - protein, 21 aa, Artificial Sequence
  ppGalNAcT substrate
AHGVTSAPDTRPAPGSTAPPA

SEQ ID NO: 9 - protein, 8 aa, Artificial Sequence
  ppGalNAcT substrate
VTPRTPPP

SEQ ID NO: 10 - protein, 7 aa, Artificial Sequence
  ppGalNAcT substrate
PTTTPLK

SEQ ID NO: 11 - protein, 11 aa, Artificial Sequence
  ppGalNAcT substrate
PTTTPITTTTK

SEQ ID NO: 12 - protein, 13 aa, Artificial Sequence
  ppGalNAcT substrate
PTTDSTTPAPTTK

SEQ ID NO: 13 - protein, 20 aa, Artificial Sequence
  ppGalNAcT substrate
PTTTPISTTTMVTPTPTPTC

SEQ ID NO: 14 - protein, 10 aa, Artificial Sequence
  ppGalNAcT substrate
DSTTPAPTTK

SEQ ID NO: 15 - protein, 16 aa, Artificial Sequence
  ppGalNAcT substrate
GTTPSPVPTTSTTSAP

SEQ ID NO: 16 - protein, 15 aa, Artificial Sequence
  ppGalNAcT substrate
PTTKDSTTPAPTTKK

SEQ ID NO: 17 - protein, 15 aa, Artificial Sequence
  ppGalNAcT substrate
PTTKDSTTPAPTTKK